(19) United States
(12) Patent Application Publication
(10) Pub. No.: US 2023/0212572 A1
(43) Pub. Date: Jul. 6, 2023

(54) Guanosine Analogues for Use in Therapeutics Polynucleotides

(71) Applicant: Roche Innovation Center Copenhagen A/S, Horsholm (DK)

(72) Inventors: Nanna Albaek, Birkerod (DK); Peter Hagedorn, Horsholm, Zealand (DK); Mette Ladefoged, Birkerod (DK); Lykke Pedersen, Kobenhavn NV (DK); Jacob Ravn, Skovlunde (DK)

(21) Appl. No.: 18/001,098

(22) PCT Filed: Jun. 8, 2021
(86) PCT No.: PCT/EP2021/065266

§ 371 Date: Dec. 8, 2022

(30) Foreign Application Priority Data
Jun. 9, 2020 (EP) 20179011.0

(51) Int. Cl. C12N 15/113

(52) U.S. Cl. CPC C12N 15/113; C12N 2310/11; C12N 2310/341; C12N 2310/315; C12N 2310/3231; C12N 2310/336; C12N 2320/53

(57) ABSTRACT

The present invention relates to a polynucleotide that comprises at least one phosphorothioate internucleoside linkage and at least one guanosine analogue comprising a guanine nucleobase analogue selected from the group consisting of: formula (I) and formula (II). Polynucleotides comprising such guanosine analogues show a relative reduced neurotoxicity compared to polynucleotides with natural guanosine.

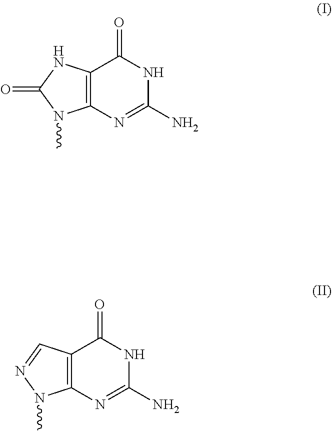

### FIELD OF INVENTION

This invention relates to polynucleotides, such as antisense oligonucleotides or siRNAs or shRNAs used as pharmaceutical active principles. More in detail, the invention relates to guanosine analogues for use in such polynucleotides.

### BACKGROUND

There is at present considerable interest in developing polynucleotides such as antisense oligonucleotide (ASOs) or siRNA therapeutics for treatment of neurological disorders. Such polynucleotides can be administered for example by intrathecal administration. The discovery of new polynucleotides therapeutics has however been hampered by a finding that a significant proportion of polynucleotides elicit neurotoxicity in animal studies (see WO2016/126995 for example). Mice administered with some polynucleotides show signs of acute neurotoxicity within 30 minutes to an hour after administration, illustrating that the toxicity is unlikely to be due to hybridization events.

WO2016/127000 reports one the use of a calcium oscillation assay to identify nucleic acid molecules, such as antisense oligonucleotides, which are likely to elicit an acute neurotoxicity in vivo, as well as methods for selecting polynucleotides which have a tolerable in vivo neurotoxicity by calculating the number of cytosine nucleotides or analogues thereof minus the number of guanosine nucleotides or analogues thereof, divided by the total length of the polynucleotide. Seela et al., Chinn. Acta 1988, 71, 1191-1198 describe using 6-chloro-7-deazapurine as well as 7-deaza-6-(methylthio)purine) as versatile candidates for glycosylation of of pyrrolo[2,3-d]pyrimidines.

Seela, F., Becher, G., Chemical Communications (1998), (18), 2017-2018 describe the introduction of 7-halogenated 7-deazapurines (pyrrolo-[2,3-d]pyrimidines A) into oligonucleotides. The purpose of this introduction into the DNA seems to be to serve as reporter groups, cleaving agents or residues useful in sequencing by mass spectrometry or by atomic force microscopy Kutyavin et al, NAR 2002, Vol 30, pp 4952-4959 discloses that 8-aza-7-deazaguanine (pyrazolo[3,4-d]pyrimidine, PPG), reduces guanine self-association of guanine-rich oligodeoxyribonucleotides, and, substitution of PPG for guanine enhances affinity, specificity, sensitivity and predictability of guanine-rich DNA probes.

Hara et al., J Org Chem reports on LNA-7-deazaguanine and LNA-8-aza-7-deazaguanine modified phosphodiester oligonucleotides, which were found to have a lower binding affinity than natural DNA, and that LNA-7-deazaguanine effectively suppresses aggregation even in a guanine-rich sequence.

This brief review of the literature shows that it remains very scarce on solving the problem of neurotoxicity observed when certain polynucleotides are administered to the central nervous system.

### OBJECTIVE OF THE INVENTION

The inventors were surprised to find that the proportion of natural, i.e. unmodified guanosine nucleobases within a polynucleotide sequence was directly correlated to the likelihood that a polynucleotide, such as an antisense oligonucleotide or siRNA, is neurotoxic. The invention provides guanosine analogues for use in therapeutic oligonucleotide thereby providing for a reduced neurotoxicity.

### SUMMARY OF INVENTION

There present invention is based upon the finding that the proportion of natural, i.e. unmodified guanosine (G) nucleobases within a polynucleotide sequence is directly correlated to the likelihood that a polynucleotide, such as an antisense oligonucleotide or siRNA, is neurotoxic. Having identified unmodified G nucleobases as the trigger for neurotoxicity, the present inventors have screened numerous G analogues, and identified certain G analogues which when used in place of the unmodified G, reduces or alleviate the neurotoxicity of a polynucleotide, illustrated by reducing neurotoxicity of phosphorothioate polynucleotide such as antisense oligonucleotides or siRNAs. In particular, the inventors have identified that the substitution of unmodified G nucleobases for 8-aza-7-deazaguanine (PPG) or 8-oxo-deoxyguanosine (8-oxo-dG) bases in a phosphorothioate antisense oligonucleotide or siRNAs alleviates neurotoxicity in vivo as well as in in vitro neurotoxicity assays.

Furthermore, the inventors have identified that whilst the incorporation of PPG bases into a polynucleotide can reduce the polynucleotides binding affinity; this is highly dependent upon the sequence context of the incorporated PPG base. The inventors have used this observation to identify short duplex and triplex motifs which are associated with a maintained effective binding affinity. Alternatively, the binding affinity may be compensated by e.g. incorporation of further high affinity nucleosides, such as LNA into an oligonucleotide.

### SEQUENCE LISTING

The sequence listing submitted with this application is hereby incorporated by reference. The antisense oligonucleotide sequence motifs listed in the sequence listing are illustrated as DNA sequences.

### FIGURE

FIG. 1 is a graph showing the rescue of acute neurotoxicity using guanine analogs in a study according to example 3.

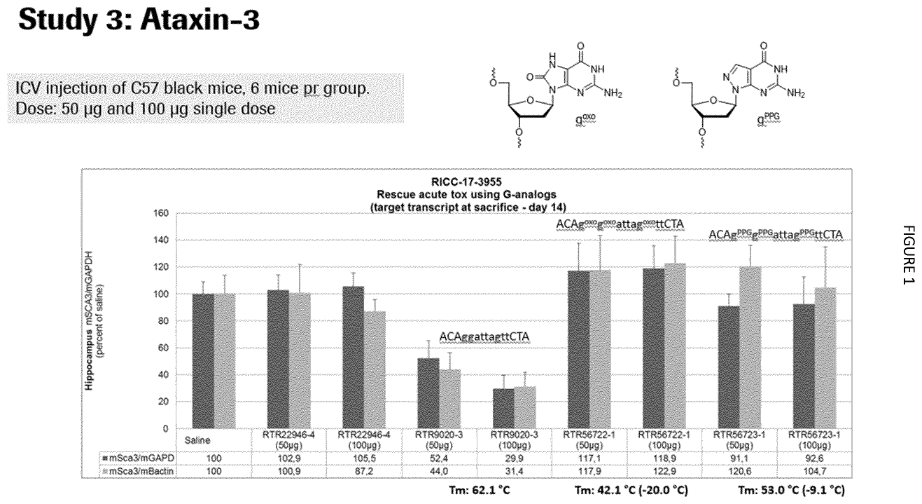

### Definitions

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides such as 2′ sugar modified nucleosides. The oligonucleotide of the invention may comprise one or more modified internucleoside linkages, such as one or more phosphorothioate internucleoside linkages.

### Antisense Oligonucleotides

The term “antisense oligonucleotide” as used herein is defined as oligonucleotides capable of modulating expression of a target gene by hybridizing to a target nucleic acid, in particular to a contiguous sequence on a target nucleic acid. Antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide.

In some embodiments, the single stranded antisense oligonucleotide of the invention may not contain non modified RNA nucleosides.

Advantageously, the antisense oligonucleotide of the invention comprises one or more modified nucleosides or nucleotides, such as 2′ sugar modified nucleosides. Furthermore, it is advantageous that the nucleosides which are not modified are DNA nucleosides.

### Contiguous Nucleotide Sequence

The term “contiguous nucleotide sequence” refers to the region of the antisense oligonucleotide which is complementary to the target nucleic acid. The term is used interchangeably herein with the term “contiguous nucleobase sequence” and the term “oligonucleotide motif sequence”. In some embodiments, all the nucleosides of the oligonucleotide constitute the contiguous nucleotide sequence. In some embodiments, the oligonucleotide comprises the contiguous nucleotide sequence, such as a F-G-F′ gapmer region, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid. In some embodiments, the nucleobase sequence of the antisense oligonucleotide is the contiguous nucleotide sequence.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. Advantageously, one or more of the modified nucleosides of the antisense oligonucleotide of the invention comprise a modified sugar moiety. The term “modified nucleoside” may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Guanosine Analogues

The term “guanine analogue” or “G analogue” as used herein refers to nucleosides or nucleotides containing the nucleobases 8-oxo-guanine (8-oxo-G) and/or 7-deaza-8-aza-guanine (PPG) of the following formulae:

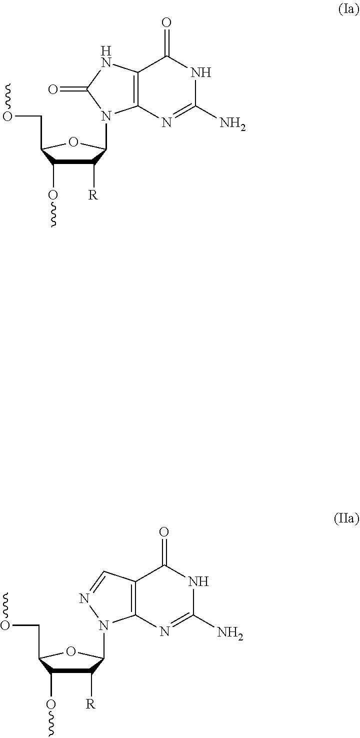

wherein R is H or OH
8-oxo-deoxyguanosine (8-oxo-dG) and/or 7-deaza-8-aza-deoxyguanosine (PPG) can also be represented more in detail as:

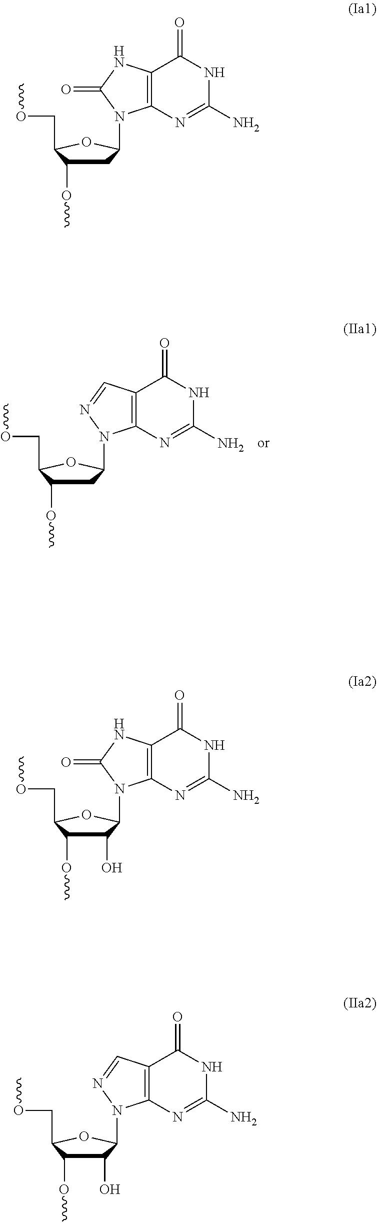

It is to be understood that this also covers guanosine analogues that comprise a sugar modification, such as the following moieties:

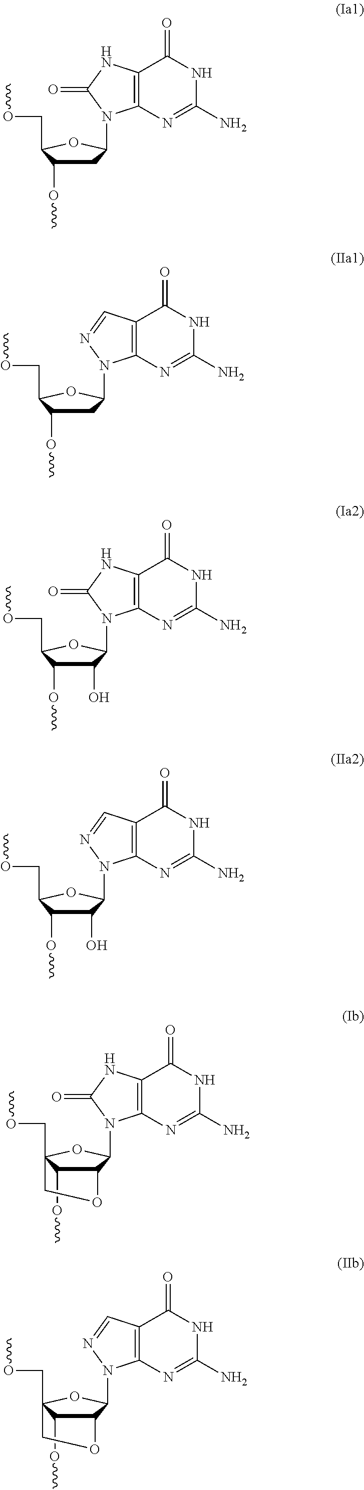

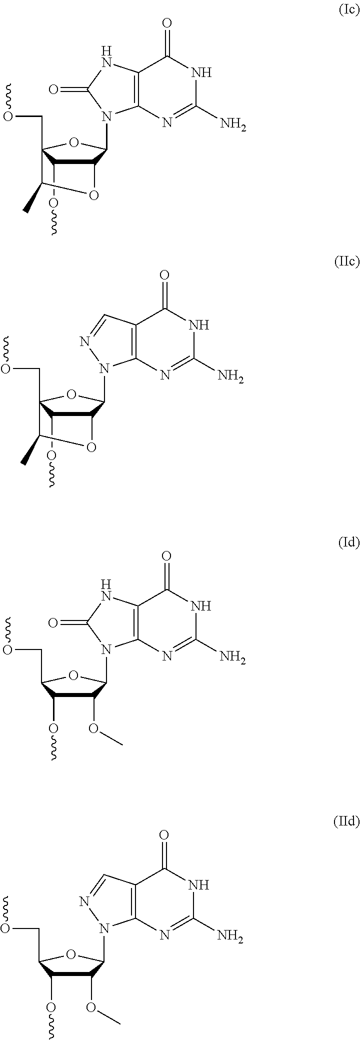

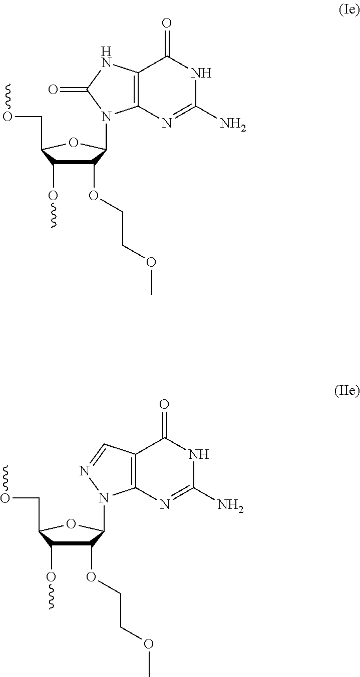

Moieties (Ia) and (IIa) can be obtained using phosphoramidites commercially available at Glen Research (Sterling, Va.).

Moieties (IIb) and (IIc) can be obtained following the synthetic routes described by Hara et al. in J. Org. Chem. 2017, 82, 25-36 and Blade et al in J. Org. Chem. 2015, 80, 5337-5343.

Moieties (Ib), (Ic), (Id) and (Ie) can be obtained from the corresponding guanosine nucleosides as described by Kannan et al in J. Org. Chem. 2011, 76, 720-723

Moieties (IId) and (IIe) can be obtained from the corresponding pentofuranosyl chloride intermediates as described by Seela et al in Nucleosides & Nucleotides (1989), 8(5-6), 789-792 and by Seela et al in Helvetica Chimica Acta (1986), 69(7), 1602-1613

### Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise one or more modified internucleoside linkages such as a one or more phosphorothioate internucleoside linkages, or one or more phoshporodithioate internucleoside linkages.

In some embodiments, at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate.

In some advantageous embodiments, all the internucleoside linkages of the contiguous nucleotide sequence of the oligonucleotide are phosphorothioate, or all the internucleoside linkages of the oligonucleotide are phosphorothioate linkages.

It is recognized that, as disclosed in EP 2 742 135, antisense oligonucleotides may comprise other internucleoside linkages (other than phosphodiester, phosphorothioate and phosphorodithioate), for example alkyl phosphonate/methyl phosphonate internucleoside, which according to EP 2 742 135 may for example be tolerated in an otherwise DNA phosphorothioate the gap region.

### Nucleobase

The term “nucleobase” includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments, the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term “modified oligonucleotide” describes an oligonucleotide comprising one or more sugar-modified nucleosides and/or modified internucleoside linkages. The term chimeric” oligonucleotide is a term that has been used in the literature to describe oligonucleotides comprising sugar modified nucleosides and DNA nucleosides. The antisense oligonucleotide of the invention is advantageously a chimeric oligonucleotide.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, guanosine analogues as described herein and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine and guanosine analogues as described herein are considered identical to a cytosine and guanosine respectively for the purpose of calculating % identity).

The term “fully complementary”, refers to 100% complementarity.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments, the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments, the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Target Nucleic Acid

According to the present invention, the target nucleic acid can be a nucleic acid which encodes a human gene, a RNA, a mRNA, and pre-mRNA, a mature mRNA or a cDNA sequence. If employing the oligonucleotide of the invention in research or diagnostics the target nucleic acid may be a cDNA or a synthetic nucleic acid derived from DNA or RNA.

### Target Sequence

The term “target sequence” as used herein refers to a sequence of nucleotides present in the target nucleic acid which comprises the nucleobase sequence which is complementary to the antisense oligonucleotide of the invention. In some embodiments, the target sequence consists of a region on the target nucleic acid with a nucleobase sequence that is complementary to the contiguous nucleotide sequence of the antisense oligonucleotide of the invention. This region of the target nucleic acid may interchangeably be referred to as the target nucleotide sequence, target sequence or target region. In some embodiments, the target sequence is longer than the complementary sequence of a single antisense oligonucleotide, and may, for example represent a preferred region of the target nucleic acid which may be targeted by several antisense oligonucleotides of the invention.

In some embodiments, the antisense oligonucleotide of the invention, or the contiguous nucleotide sequence thereof, is complementary, such as fully complementary to a target sequence.

The antisense oligonucleotide of the invention comprises a contiguous nucleotide sequence which is complementary to and hybridizes to the target nucleic acid, such as a target sequence described herein.

The target sequence to which the antisense oligonucleotide is complementary to generally comprises a contiguous nucleobases sequence of at least 10 nucleotides. The contiguous nucleotide sequence is between 10 to 30 nucleotides in length, such as 12 to 30, such as 14 to 20, such as 15 to 18 contiguous nucleotides in length, such as 15, 16, 17 contiguous nucleotides in length.

In some embodiments, the antisense oligonucleotide of the invention is fully complementary to the target sequence across the full length of the antisense oligonucleotide.

### Target Cell

The term a “target cell” as used herein refers to a cell which is expressing the target nucleic acid. In some embodiments, the target cell may be in vivo or in vitro. In some embodiments, the target cell is a mammalian cell such as a rodent cell, such as a mouse cell or a rat cell, or a primate cell such as a monkey cell or a human cell.

Typically, the target cell expresses the target mRNA, such as the target pre-mRNA or target mature mRNA. For experimental evaluation a target cell may be used which expresses a nucleic acid which comprises a target sequence.

The antisense oligonucleotides of the invention are typically capable of inhibiting the expression of a target nucleic acid in a cell which is expressing the target nucleic acid (a target cell), for example either in vivo or in vitro.

The contiguous sequence of nucleobases of the antisense oligonucleotide of the invention is complementary, such as fully complementary to the target nucleic acid, as measured across the length of the antisense oligonucleotide, optionally excluding nucleotide based linker regions which may link the antisense oligonucleotide to an optional functional group such as a conjugate, or other non-complementary terminal nucleotides (e.g. region D′ or D″). The target nucleic acid may for example be a messenger RNA, such as a mature mRNA or a pre-mRNA, which encodes a given protein.

### Naturally Occurring Variant

The term “naturally occurring variant” refers to variants of a gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian target nucleic acid.

### Inhibition of Expression

The term “Inhibition of expression” as used herein is to be understood as an overall term for an oligonucleotide's ability to inhibit the amount or the activity of a given protein in a target cell. Inhibition of activity may be determined by measuring the level of a target pre-mRNA or target mRNA, or by measuring the level of the target gene or target gene activity in a cell. Inhibition of expression may therefore be determined in vitro or in vivo. Inhibition of a target expression may also be determined by measuring activity or protein level.

Typically, inhibition of expression is determined by comparing the inhibition of activity due to the administration of an effective amount of the antisense oligonucleotide to the target cell and comparing that level to a reference level obtained from a target cell without administration of the antisense oligonucleotide (control experiment), or a known reference level (e.g. the level of expression prior to administration of the effective amount of the antisense oligonucleotide, or a predetermine or otherwise known expression level).

For example a control experiment may be an animal or person, or a target cell treated with a saline composition or a reference oligonucleotide (often a scrambled control).

The term inhibition or inhibit may also be referred as down-regulate, reduce, suppress, lessen, lower, the expression of a given geneA.

The inhibition of expression may occur e.g. by degradation of pre-mRNA or mRNA (e.g. using RNaseH recruiting oligonucleotides, such as gapmers).

### High Affinity Modified Nucleosides

A high affinity modified nucleoside is a modified nucleotide which, when incorporated into the antisense oligonucleotide enhances the affinity of the antisense oligonucleotide for its complementary target, for example as measured by the melting temperature (Tm). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The antisense oligonucleotide of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance. In some embodiments of the invention the term “sugar modification” means “ribose modification” or “modified ribose” or a “modification of the ribose”.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside, also known as 2′ sugar modification, is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′-4′ biradical bridged) nucleosides.

Indeed, much focus has been spent on developing 2′ sugar substituted nucleosides, and numerous 2′ substituted nucleosides have been found to have beneficial properties when incorporated into antisense oligonucleotides. For example, the 2′ modified sugar may provide enhanced binding affinity and/or increased nuclease resistance to the antisense oligonucleotide. Examples of 2′ substituted nucleosides are 2′-O-alkyl-RNA/DNA, 2′-O-methyl-RNA/DNA, 2′-alkoxy-RNA/DNA, 2′-O-methoxyethyl-RNA/DNA (MOE), 2′-amino-DNA, 2′-Fluoro-RNA/DNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

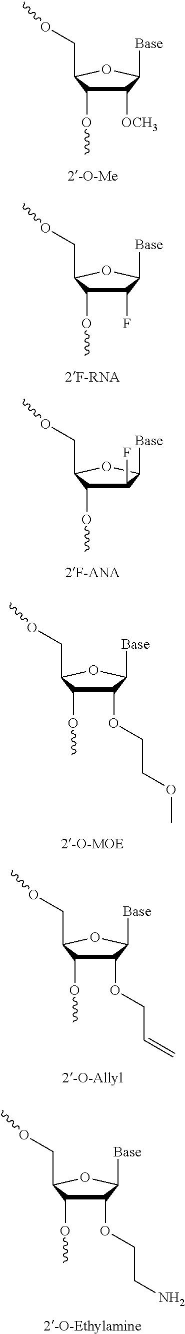

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an antisense oligonucleotide for a complementary RNA or DNA molecule. This can be routinely determined by measuring the melting temperature of the antisense oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non-limiting, exemplary LNA nucleosides are disclosed in Scheme 1.

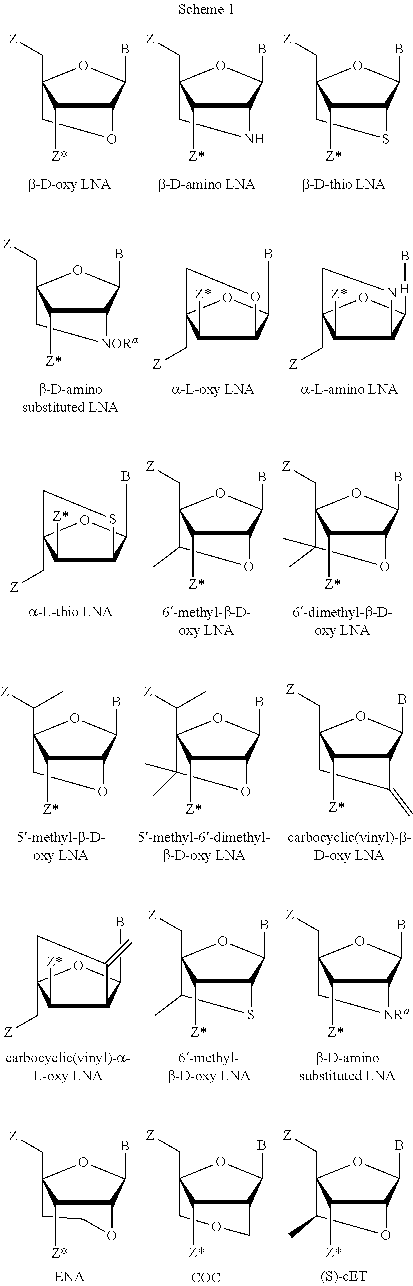

Wherein B is a natural or non-natural (modified) nucleobase and Z is an internucleoside linkage to an adjacent nucleoside or a 5′-terminal group and Z* is an internucleoside linkage to an adjacent nucleoside or a 3′-terminal group;

It will be recognized than, unless specified, the LNA nucleosides may be in the beta-D or alpha-L stereoisoform.

Exemplary Nucleosides, with HELM Annotation

### DNA Nucleosides

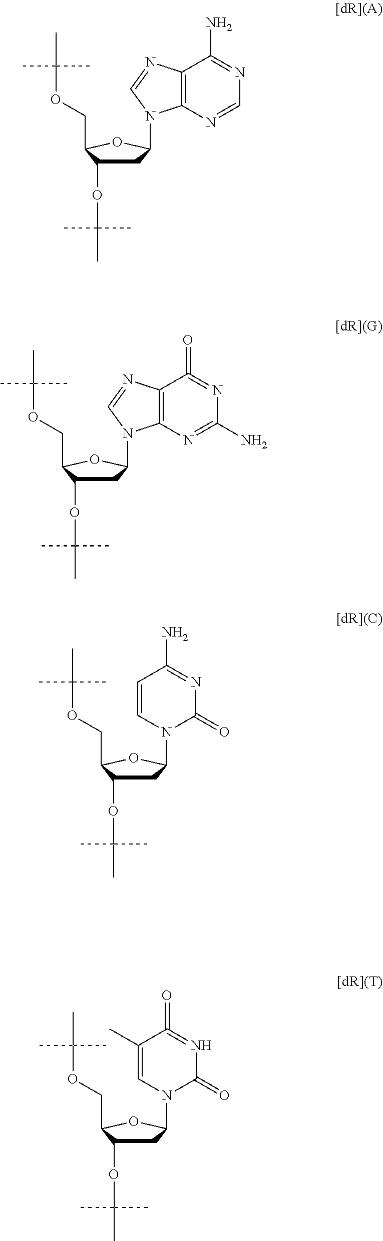

### Beta-D-oxy-LNA Nucleosides

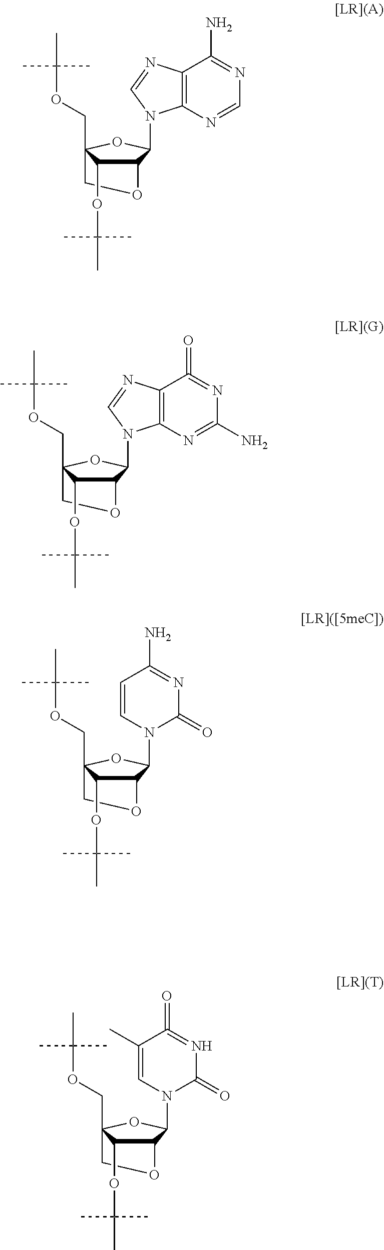

### 2′-O-methyl Nucleosides

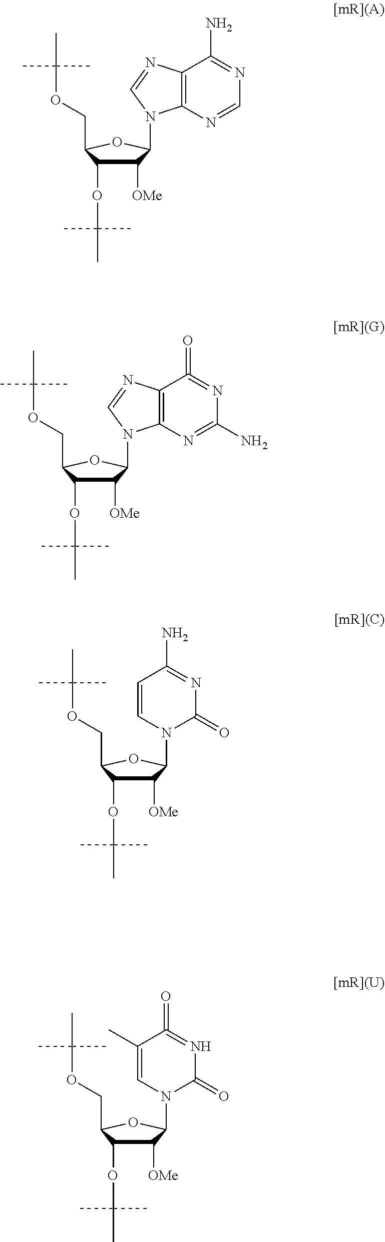

Exemplary phosphorothioate Internucleoside Linkage with HELM Annotation

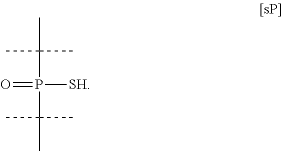

The dotted lines represent the covalent bond between each nucleoside and the 5′ or 3′ phosphorothioate internucleoside linkages. At the 5′ terminal nucleoside, the 5′ dotted lines represent a bond to a hydrogen atom (forming a 5′ terminal —OH group). At the 3′ terminal nucleoside, the 3′ dotted lines represent a bond to a hydrogen atom (forming a 3′ terminal —OH group).

### RNase H Activity and Recruitment

The RNase H activity of an antisense oligonucleotide refers to its ability to recruit RNase H when in a duplex with a complementary RNA molecule. WO 01/23613 provides in vitro methods for determining RNase H activity, which may be used to determine the ability to recruit RNase H. Typically an antisense oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a antisense oligonucleotide having the same base sequence as the modified oligonucleotide being tested, but containing only DNA monomers with phosphorothioate linkages between all monomers in the antisense oligonucleotide, and using the methodology provided by Example 91-95 of W 001/23613 (hereby incorporated by reference). For use in determining RNase H activity, recombinant human RNase H1 is available from Creative Biomart® (Recombinant Human RNASEH1 fused with His tag expressed in E. coli).

### Gapmer

The antisense oligonucleotide of the invention, or contiguous nucleotide sequence thereof, may be a gapmer, also termed gapmer antisense oligonucleotide or gapmer designs. The gapmers are commonly used to inhibit a target nucleic acid via RNase H mediated degradation. A gapmer comprises at least three distinct structural regions a 5′-flank, a gap and a 3′-flank, F-G-F′ in the ‘5->3’ orientation. The “gap” region (G) comprises a stretch of contiguous DNA nucleotides which enable the gapmer to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the gapmer for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′. The overall length of the gapmer design F-G-F′ may be, for example 12 to 32 nucleosides, such as 13 to 24, such as 14 to 22 nucleosides, Such as from 14 to 17, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

In an aspect of the invention the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region between 6 and 16 nucleosides which are capable of recruiting RNase H.

Regions F, G and F′ are further defined below and can be incorporated into the F-G-F′ formula.

### LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments, the LNA gapmer is of formula: [LNA]₁₋₅-[region G]-[LNA]₁₋₅, wherein region G is or comprises a region of contiguous DNA nucleosides which are capable of recruiting RNase H.

### MOE Gapmers

A MOE gapmers is a gapmer wherein regions F and F′ consist of MOE nucleosides. In some embodiments, the MOE gapmer is of design [MOE]₁₋₈-[Region G]₅₋₁₆-[MOE]₁₋₈, such as [MOE]₂₋₇-[Region G]₆₋₁₄-[MOE]₂₋₇, such as [MOE]₃₋₆-[Region G]₈₋₁₂-[MOE]₃₋₆, wherein region G is as defined in the Gapmer definition. MOE gapmers with a 5-10-5 design (MOE-DNA-MOE) have been widely used in the art.

### Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleoside. In some embodiments, wherein at least one of region F and F′, or both region F and F′ comprise at least one LNA nucleoside, the remaining nucleosides of region F and F′ are independently selected from the group consisting of MOE and LNA. In some embodiments, wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

### Alternating Flank Gapmers

Flanking regions may comprise both LNA and DNA nucleoside and are referred to as “alternating flanks” as they comprise an alternating motif of LNA-DNA-LNA nucleosides. Gapmers comprising such alternating flanks are referred to as “alternating flank gapmers”. “Alternative flank gapmers” are thus LNA gapmer oligonucleotides where at least one of the flanks (F or F′) comprises DNA in addition to the LNA nucleoside(s). In some embodiments, at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

### Region D′ or D″ in an Antisense Oligonucleotide

The antisense oligonucleotide of the invention may In some embodiments, comprise or consist of the contiguous nucleotide sequence of the oligonucleotide which is complementary to the target nucleic acid, such as a gapmer region F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the antisense oligonucleotide and region D′ or D″ constitute a separate part of the antisense oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments, the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single antisense oligonucleotide.

In one embodiment, the antisense oligonucleotide of the invention comprises a region D′ and/or D″ in addition to the contiguous nucleotide sequence which constitutes the gapmer.

In some embodiments, the antisense oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments, the internucleoside linkage positioned between region D′ and region F is a phosphodiester linkage. In some embodiments, the internucleoside linkage positioned between region F′ and region D″ is a phosphodiester linkage.

### Conjugate

The term conjugate as used herein refers to an antisense oligonucleotide which is covalently linked to a non-nucleotide moiety (conjugate moiety or region C or third region). The conjugate moiety may be covalently linked to the antisense oligonucleotide, optionally via a linker group, such as region D′ or D″.

Antisense oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the antisense oligonucleotide directly or through a linking moiety (e.g. linker or tether). Linkers serve to covalently connect a third region, e.g. a conjugate moiety (Region C), to a first region, e.g. an antisense oligonucleotide or contiguous nucleotide sequence complementary to the target nucleic acid (region A).

In some embodiments, of the invention the conjugate or antisense oligonucleotide conjugate of the invention may optionally, comprise a linker region (second region or region B and/or region Y) which is positioned between the antisense oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment, the biocleavable linker is susceptible to S1 nuclease cleavage. In some embodiments, the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Region Y refers to linkers that are not necessarily biocleavable but primarily serve to covalently connect a conjugate moiety (region C or third region), to an antisense oligonucleotide (region A or first region). The region Y linkers may comprise a chain structure or an oligomer of repeating units such as ethylene glycol, amino acid units or amino alkyl groups. The antisense oligonucleotide conjugates of the present invention can be constructed of the following regional elements A-C, A-B-C, A-B-Y-C, A-Y-B-C or A-Y-C. In some embodiments, the linker (region Y) is an amino alkyl, such as a C2-C36 amino alkyl group, including, for example C6 to C12 amino alkyl groups. In some embodiments, the linker (region Y) is a C6 amino alkyl group. In some embodiments, the linker is NA.

### SiRNA

A “small interfering” or “short interfering RNA” or siRNA is a RNA duplex of nucleotides that is targeted to a gene interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

As used herein, an “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a conventional shRNA forms a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The terms “hairpin” and “fold-back” structures are also used herein to refer to stem-loop structures.

Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

“RNAi-expressing construct” or “RNAi construct” is a generic term that includes nucleic acid preparations designed to achieve an RNA interference effect. An RNAi-expressing construct comprises an RNAi molecule that can be cleaved in vivo to form an siRNA or a mature shRNA. For example, an RNAi construct is an expression vector capable of giving rise to an siRNA or a mature shRNA in vivo. Non-limiting examples of vectors that may be used in accordance with the present invention are described herein, for example, in section 4.6. Exemplary methods of making and delivering long or short RNAi constructs can be found, for example, in WO01/68836 and WO01/75164.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

In a first aspect, the invention relates to a polynucleotide that comprises:
at least one phosphorothioate internucleoside linkage and
at least a guanosine analogue comprising a guanine analogue selected from the group consisting of:

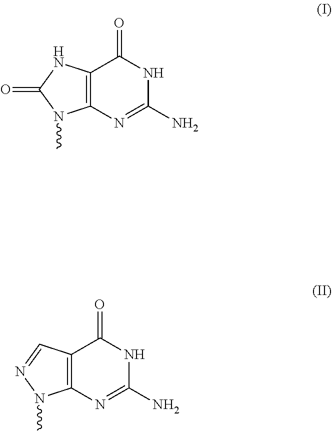

The polynucleotide can be single stranded, such as for example an antisense oligonucleotide.

The polynucleotide can be double stranded such as siRNA or shRNA.

The polynucleotide can comprise one or more 2′ sugar modified nucleosides. Such 2′ sugar modified nucleoside(s) can be independently selected from the group consisting of locked nucleic acids and 2′ sugar substituted nucleosides.

The 2′ sugar modified nucleosides can be locked nucleic acids selected from the group consisting of:

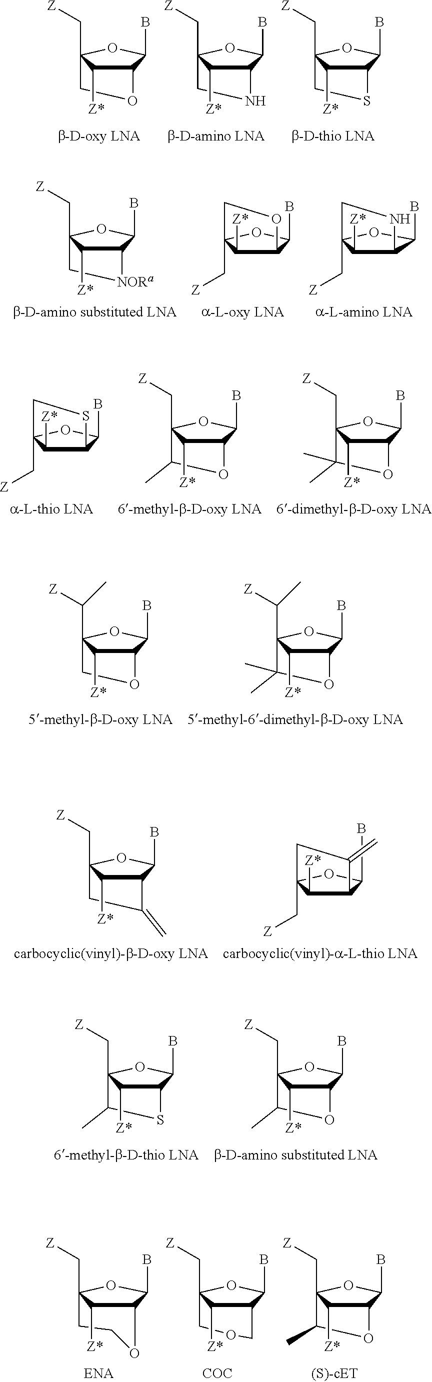

wherein B is a natural or modified nucleobase and Z is an internucleoside linkage to an adjacent nucleoside or a 5′-terminal group and Z* is an internucleoside linkage to an adjacent nucleoside or a 3′-terminal group.

The 2′ sugar modified nucleosides can be selected from the group consisting of:

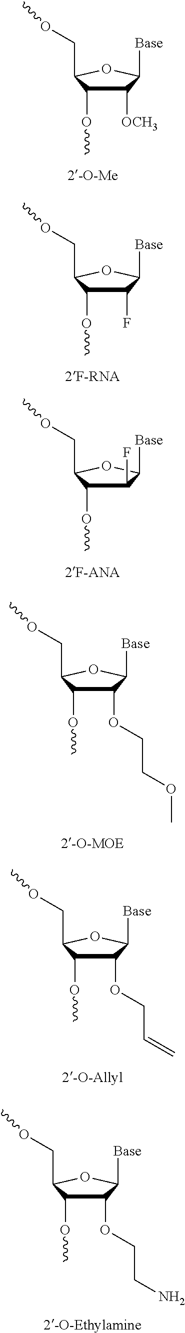

The guanosine analogue can be selected from the group consisting of:

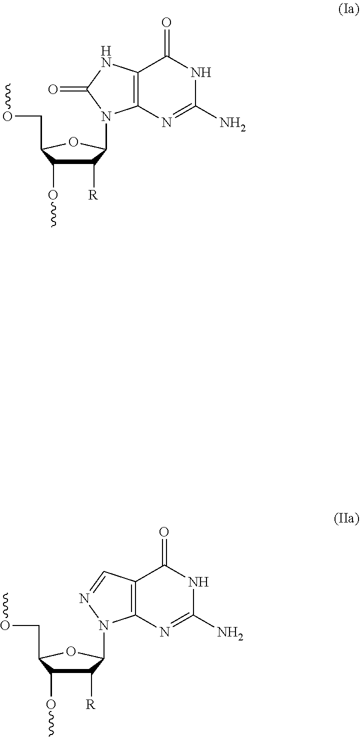

wherein R is H or OH

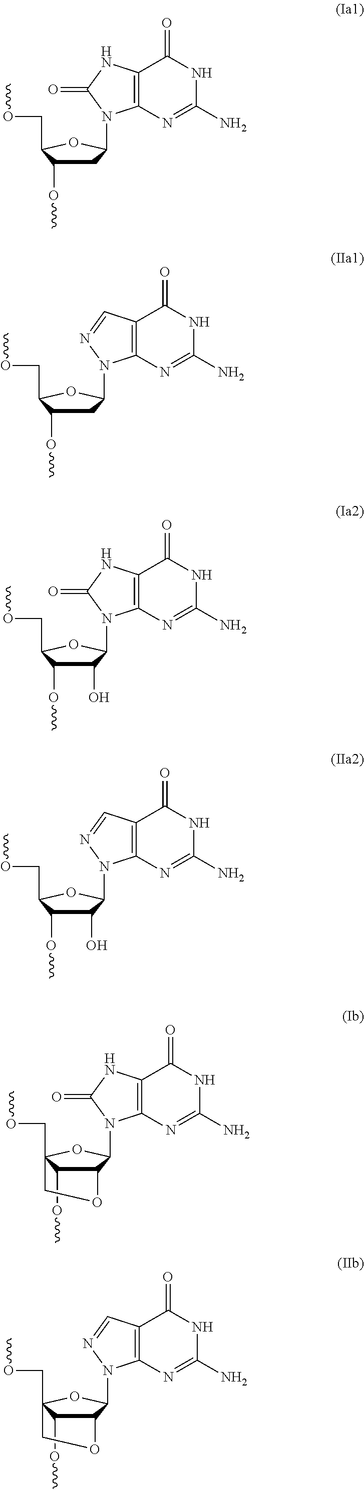

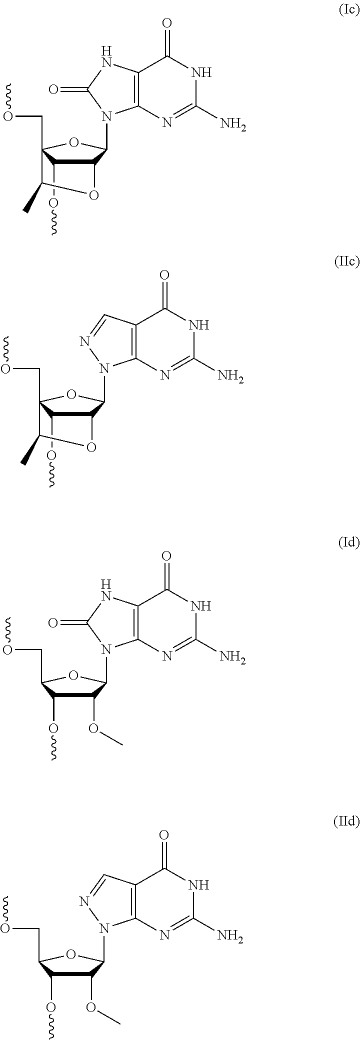

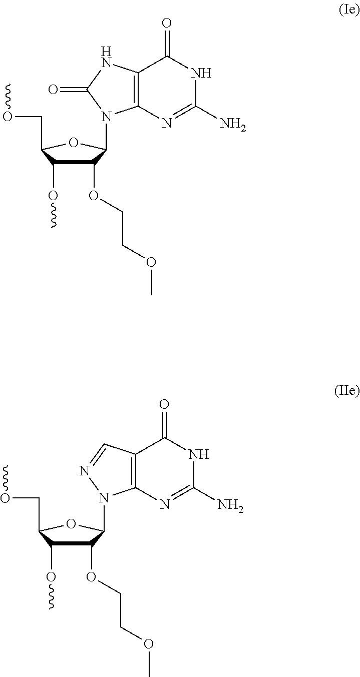

In an embodiment of the invention the guanosine analogue is (la).

In an embodiment of the invention the guanosine analogue is (Ib).

In an embodiment of the invention the guanosine analogue is (IIa).

In an embodiment of the invention the guanosine analogue is (IIb).

In an embodiment of the invention the guanosine analogue is (Ic).

In an embodiment of the invention the guanosine analogue is (IIc).

In an embodiment of the invention the guanosine analogue is (Id).

In an embodiment of the invention the guanosine analogue is (IId).

In an embodiment of the invention the guanosine analogue is (Ie).

In an embodiment of the invention the guanosine analogue is (IIe).

According to the invention, the antisense oligonucleotide can be a gapmer.

According to the invention, the polynucleotide can comprise at least one additional nucleoside that have a modified ribose and wherein the wherein the ribose modification is selected from the group consisting of locked nucleic acid or other 2′ modification.

In an embodiment of the invention, the guanosine analogue is in the gap region of the gapmer and is of formula Ia or IIb and wherein R is H.

In an embodiment of the invention, the guanosine analogue is not in the flank of the gapmer.

In an embodiment of the invention, the polynucleotide comprises one guanosine analogue.

In an embodiment of the invention, the polynucleotide comprises two guanosine analogues.

In an embodiment of the invention the polynucleotide comprises three guanosine analogues.

In an embodiment of the invention the polynucleotide comprises no natural guanosine.

In an embodiment of the invention the polynucleotide is an antisense selected from the group consisting of:

(SEQ ID No. 4)
CTCAacttg^{OXO}ctttaAT;
(SEQ ID No. 5)
CTCAtacttg^{N}ctttaAT;
(SEQ ID No. 6)
CTCAtacttg^{PPG}ctttaAT;
(SEQ ID No. 9)
CTAcatctcatactTgC;
(SEQ ID No. 10)
CTAcatctcatactTg^{PPG}C;
(SEQ ID No. 11)
CTAcatctcatactTg^{OXO}C;
(SEQ ID No. 13)
CTAcatctcatactTg^{N}c;
(SEQ ID No. 15)
ACAg^{OXO}g^{OXO}attag^{OXO}ttCTA;
and
(SEQ ID No. 16)
ACAg^{PPG}g^{PPG}attag^{PPG}ttCTA;

wherein capital letters in these sequences indicate nucleoside that have an LNA modified ribose, all LNA C are 5-methyl cytosine and small letters in these sequences indicate DNA,
g^{PPG} is 7-Deaza-8-aza-deoxyguanosine,
g^{N} is 8-amino-dG, and
g^{oxo} is 8-Oxo-deoxyguanosine.
In an embodiment of the invention the antisense oligonucleotide is CTCAacttg^{oxo}ctttaAT (SEQ ID No. 4).
In an embodiment of the invention the antisense oligonucleotide is CTCAtacttg^{C}ctttaAT (SEQ ID No. 5).
In an embodiment of the invention the antisense oligonucleotide is CTCAtacttg^{PPG}ctttaAT (SEQ ID No. 6).
In an embodiment of the invention the antisense oligonucleotide is CTAcatctcatactTgC (SEQ ID No. 9).
In an embodiment of the invention the antisense oligonucleotide is CTAcatctcatactTg^{PPG}C (SEQ ID No. 10).
In an embodiment of the invention the antisense oligonucleotide is CTAcatctcatactTg^{oxo}C (SEQ ID No. 11).
In an embodiment of the invention the antisense oligonucleotide is CTAcatctcatactTg^{N}C (SEQ ID No. 13).
In an embodiment of the invention the antisense oligonucleotide is ACAg^{oxo}g^{oxo}attag^{oxo}ttCTA (SEQ ID No. 15).
In an embodiment of the invention the antisense oligonucleotide is ACAg^{PPG}g^{PPG}attag^{PPG}ttCTA (SEQ ID No. 16),
wherein capital letters in the above sequences indicate nucleoside that have an LNA modified ribose, all LNA C are 5-methyl cytosine and small letters in these sequences indicate DNA, and
and wherein:
g^{PPG} is 7-Deaza-8-aza-deoxyguanosine,
g^{N} is 8-amino-dG, and
g^{oxo} is 8-Oxo-deoxyguanosine.

The invention also relates to a polynucleotide according to the invention for use as a medicament. It can be used for the administration to the central nervous system, or for treatment of a neurological disorder, such as a CNS disorder selected from the group consisting of amyotrophic lateral sclerosis (ALS), Angelman's, Alzheimer's disease, Aneurysm, Back pain, Bell's palsy, Birth defects of the brain and spinal cord, Brain injury, Brain tumor, Cerebral palsy, Chronic fatigue syndrome, Concussion, Dementia, Disk disease of neck and lower back, Dizziness, Epilepsy, Guillain-Barré syndrome, Headaches and migraines, Multiple sclerosis, Muscular dystrophy, Neuralgia, Neuropathy, Neuromuscular and related diseases, Parkinson's disease, Psychiatric conditions (severe depression, obsessive-compulsive disorder), Scoliosis, Seizures, Spinal cord injury, Spinal deformity and disorders, Spine tumor, Stroke and Vertigo.

The polynucleotide according to the invention can be administered via intrathecal injection.

The polynucleotide according to the invention can be used as a medicament to treat a medical condition wherein modulation of Ube3A is beneficial, such as for the treatment of Angelman's.

The polynucleotide according to the invention can be used as a medicament to treat a medical condition wherein modulation of ATXN2 is beneficial.

The polynucleotide according to the invention can be used as a medicament to treat a medical condition wherein modulation of ATXN3 is beneficial.

The polynucleotide of the invention can be used as a medicament wherein reduced neurotoxicity is needed.

The invention also relates to a method for synthesizing a polynucleotide of the invention with a reduced toxicity, said method comprising coupling a nucleotide monomer, such as a phosphoramidite to a further nucleotide, or an oligonucleotide, wherein the nucleotide monomer comprises a guanosine analogue as defined hereinabove.

The invention also relates to a method for selecting a polynucleotide with a reduced toxicity over a reference polynucleotide, wherein the polynucleotide comprises at least one phosphorothioate internucleoside linkage and wherein the reference polynucleotide and the less neurotoxic antisense oligonucleotide have the same nucleotide sequence and comprise at least one guanosine with the difference that the less neurotoxic polynucleotide comprises at least a guanosine analogue compared to the reference polynucleotide.

The invention also relates to the use of a compound containing a guanosine analogue selected from the group consisting of (Ia), (Ib), (Ic), (Id), (Ie), (IIa), (IIb), (IIc), (IId) and (IIe) in the manufacture of an oligonucleotide antisense or shRNA or siRNA.

The invention also relates to a conjugate comprising the polynucleotide according to the invention, and at least one conjugate moiety covalently attached to said polynucleotide.

The invention also relates to a pharmaceutically acceptable salt of the polynucleotide of the invention, or a conjugate as defined hereinabove.

In some embodiments, the pharmaceutically acceptable salt is a sodium salt or a potassium salt.

A pharmaceutical composition comprising the antisense oligonucleotide according to the invention, or the conjugate according to the invention, or the pharmaceutically acceptable salt according to the invention, and a pharmaceutically acceptable diluent, solvent, carrier, salt and/or adjuvant.

The present invention also relates to a method for up-regulating Ube3a expression in a target cell which is expressing Ube3a-ATS, said method comprising administering the antisense oligonucleotide according to the invention which targets Ube3a-ATS, or the conjugate according to the invention, or the pharmaceutically acceptable salt according to the invention, in an effective amount to said cell.

In some embodiments, said method is an in vivo method or an in vitro method.

The invention also relates to a method for treating or preventing neurological disorder in a subject, such as a human, who is suffering from or is likely to suffer neurological disorders, comprising administering a therapeutically or prophylactically effective amount of the polynucleotide according to the invention, or the conjugate according to the invention, or the pharmaceutically acceptable salt according to the invention, such as to prevent or alleviate the neurological disorder.

The antisense oligonucleotide that comprises at least one phosphorothioate internucleoside linkage. It is to be understood that the antisense oligonucleotide of the invention can comprises more than one phosphorothioate. The oligonucleotides of the invention may therefore comprise one or more modified internucleoside linkages such as a one or more phosphorothioate internucleoside linkages, or one or more phosphorothioate internucleoside linkages. In some embodiments, at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate.

In an embodiment, the locked nucleic acid is selected from the group consisting of:

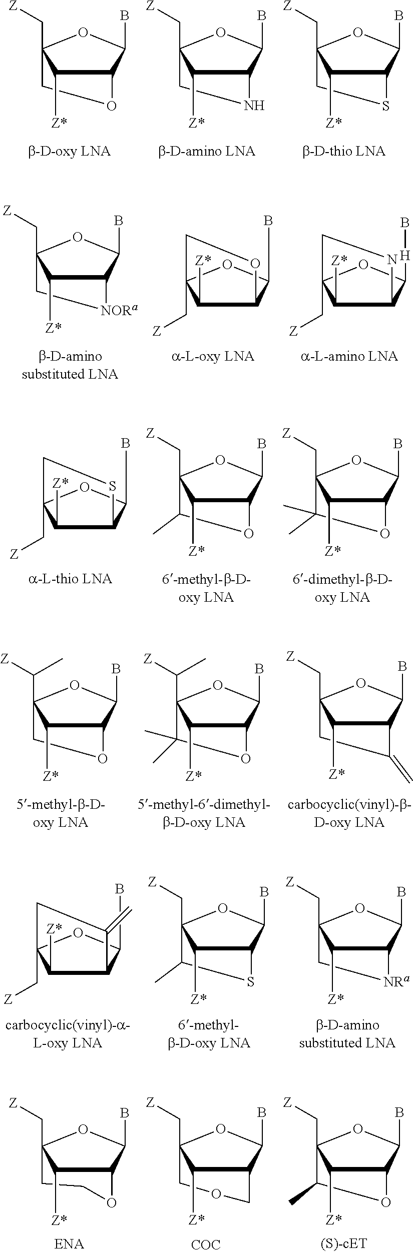

wherein B is a natural or modified nucleobase and Z is an internucleoside linkage to an adjacent nucleoside or a 5′-terminal group and Z* is an internucleoside linkage to an adjacent nucleoside or a 3′-terminal group.

In an embodiment of the antisense of the invention, the 2′ modification can be selected from the group consisting of:

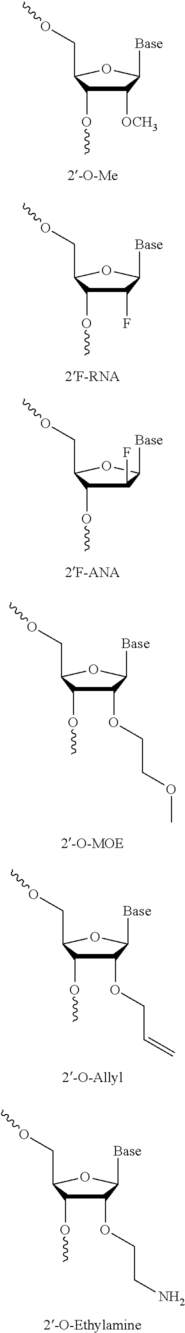

In an embodiment of the invention, the antisense oligonucleotides can comprise at least one additional nucleoside that have a modified ribose and wherein the wherein the ribose modification is selected from the group consisting of locked nucleic acid or 2′ modification.

In an embodiment of the invention, the antisense oligonucleotides is a gapmer as defined hereinabove.

In some embodiments, the antisense oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

wherein F is the Flank and G the gap.

In some embodiments, the internucleoside linkage positioned between region D′ and region F is a phosphodiester linkage. In some embodiments, the internucleoside linkage positioned between region F′ and region D″ is a phosphodiester linkage.

In the antisense oligonucleotides of the invention, the guanosine analogue can be in the gap of the gapmer. In some embodiments according to the invention, the guanosine analogue is not in the flank of the gapmer.

The antisense oligonucleotides of the invention can comprise one guanosine analogue. The antisense oligonucleotides of the invention can comprise two guanosine analogues. The antisense oligonucleotides of the invention can comprise three guanosine analogues.

In some embodiments of the invention, the antisense oligonucleotide comprises no natural guanosine.

Because it provides a relative lower neurotoxicity than conventional antisense oligonucleotides, the antisense oligonucleotide of the invention can be used as a medicament. It can be administered to the central nervous system, for example via intrathecal injection.

The antisense oligonucleotide according to the invention can be used as a medicament to treat a medical condition wherein modulation of Ube3A is beneficial, for example Angelman's syndrome.

The antisense oligonucleotide according to the invention can be used as a medicament to treat a medical condition wherein modulation of ATXN2 is beneficial, for example spinocerebellar ataxia type II (SCA2) and amyotrophic lateral sclerosis (ALS).

The antisense oligonucleotide according to the invention can be used as a medicament to treat a medical condition wherein modulation of ATXN3 is beneficial, for example spinocerebellar ataxia type 3 (SCA3).

The antisense oligonucleotide according to the invention can be used as a medicament to treat a medical condition wherein reduced neurotoxicity of the medicament is needed.

The invention further relates to a method for designing a less neurotoxic antisense oligonucleotide of the invention, over a reference compound, wherein the reference compound and the less neurotoxic antisense oligonucleotide have the same nucleotide sequence and comprise at least one guanosine with the difference that the less neurotoxic antisense oligonucleotide comprises at least a guanosine analogue compared to the reference compound.

The invention further relates to the use of guanosine analogue selected from the group consisting of (Ia), (Ib), (Ic), (Id), (Ie), (IIa), (IIb), (IIc), (IId) and (IIe) in the manufacture of an oligonucleotide.

The invention also relates to a conjugate comprising the antisense oligonucleotide of the invention, and at least one conjugate moiety covalently attached to said antisense oligonucleotide.

The invention further relates to a pharmaceutically acceptable salt of the antisense oligonucleotide of the invention or a conjugate thereof. In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

The antisense oligonucleotide of the invention can be under the form of a pharmaceutically acceptable salt such as sodium salt or a potassium salt.

The invention also relates to a pharmaceutical composition comprising the antisense oligonucleotide of the invention or of a conjugate or of a pharmaceutically acceptable salt and one or more pharmaceutically acceptable diluent, solvent, carrier, salt and/or adjuvant.

The invention also relates to a method for inhibiting Ube3a expression in a target cell, which is expressing Ube3a, said method comprising administering the antisense oligonucleotide of the invention, or a conjugate, or pharmaceutically acceptable salt thereof, in an effective amount to said cell. Said method can be an in vivo method or an in vitro method.

The invention further relates to a method for treating or preventing neurological disorders in a subject, such as a human, who is suffering from or is likely to suffer neurological disorders, comprising administering a therapeutically or prophylactically effective amount of the antisense oligonucleotide of the invention, or the conjugate according of the invention, or the pharmaceutically acceptable salt of the invention, such as to prevent or alleviate neurological disorders.

### The Antisense Oligonucleotide

In some embodiments, the antisense oligonucleotide of the invention is capable of modulating the expression of the target by inhibiting or down-regulating it. Preferably, such modulation produces an inhibition of expression of at least 20% compared to the normal expression level of the target, more preferably at least 30%, at least 40%, at least 50% inhibition compared to the normal expression level of the target. In some embodiments, antisense oligonucleotides of the invention may be capable of inhibiting expression levels of a target mRNA by at least 60% or 70% in vitro following application of 0.031 μM, 0.1 μM, and 0.4 μM antisense oligonucleotide to target cells. In some embodiments, antisense oligonucleotides of the invention may be capable of inhibiting expression levels of target gene by at least 50% in vitro following application of 0.031 μM, 0.1 μM, and 0.4 μM oligonucleotide to target cells. Suitably, the Examples provide assays which may be used to measure target RNA or protein inhibition. The target modulation is triggered by the hybridization between a contiguous nucleotide sequence of the antisense oligonucleotide and the target nucleic acid. In some embodiments, the antisense oligonucleotide of the invention comprises mismatches between the antisense oligonucleotide and the target nucleic acid. Despite mismatches hybridization to the target nucleic acid may still be sufficient to show a desired modulation of target gene expression. Reduced binding affinity resulting from mismatches may advantageously be compensated by increased number of nucleotides in the antisense oligonucleotide and/or an increased number of modified nucleosides capable of increasing the binding affinity to the target, such as 2′ sugar modified nucleosides, including LNA, present within the antisense oligonucleotide sequence.

An aspect of the present invention relates to an antisense oligonucleotide, which comprises a contiguous nucleotide sequence of 10 to 30 nucleotides in length with at least 90% complementarity to a target pre-mRNA or mRNA or a transcript variant derived therefrom.

In some embodiments, the antisense oligonucleotide comprises a contiguous sequence of 10 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the antisense oligonucleotide of the invention, or contiguous nucleotide sequence thereof is fully complementary (100% complementary) to a region of the target nucleic acid, or in some embodiments, may comprise one or two mismatches between the antisense oligonucleotide and the target nucleic acid.

In some embodiments, the antisense oligonucleotide comprises a contiguous nucleotide sequence, which is fully (or 100%) complementary, to a region of the target nucleic acid.

The antisense oligonucleotide of the invention comprises a contiguous nucleotide sequence, which is complementary to or hybridizes to a region of the target nucleic acid, such as a target sequence described herein.

The target nucleic acid sequence to which the therapeutic antisense oligonucleotide is complementary or hybridizes to generally comprises a stretch of contiguous nucleobases of at least 10 nucleotides. The contiguous nucleotide sequence is between 12 to 70 nucleotides, such as 12 to 50, such as 13 to 30, such as 14 to 25, such as 14 to 20 contiguous nucleotides.

In some embodiments, the antisense oligonucleotide of the invention or contiguous nucleotide sequence thereof, comprises or consists of 10 to 30 nucleotides in length, such as from 12 to 25, such as 11 to 22, such as from 12 to 20, such as from 14 to 18 or 14 to 16 contiguous nucleotides in length.

In some embodiments, the antisense oligonucleotide or contiguous nucleotide sequence thereof comprises or consists of 22 or less nucleotides, such as 20 or less nucleotides, such as 18 or less nucleotides, such as 14, 15, 16 or 17 nucleotides. It is to be understood that any range given herein includes the range endpoints. Accordingly, if an antisense oligonucleotide is said to include from 10 to 30 nucleotides, both 10 and 30 nucleotides are included.

In some embodiments, the contiguous nucleotide sequence comprises or consists of 10, 11, 12, 13, 14, 15, 16, 17, 18, 19, 20, 21, 22, 23, 24, 25, 26, 27, 28, 29 or 30 contiguous nucleotides in length.

In some embodiments, the contiguous nucleotide sequence comprises or consists of 12, 13, 14, 15, 16, 17, 18, 19, 20, 21, or 22, contiguous nucleotides in length.

In advantageous embodiments, the antisense oligonucleotide comprises one or more sugar modified nucleosides, such as one or more 2′ sugar modified nucleosides, such as one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In some embodiments, the contiguous nucleotide sequence comprises LNA nucleosides.

In some embodiments, the contiguous nucleotide sequence comprises LNA nucleosides and DNA nucleosides.

In some embodiments, the contiguous nucleotide sequence comprises LNA nucleosides and DNA nucleosides and 2′-O-methyl RNA nucleosides.

In some embodiments, the contiguous nucleotide sequence comprises LNA nucleosides and DNA nucleosides and 2′-O-methyl RNA nucleosides, and the internucleoside linkages between each of the nucleosides of the contiguous nucleotide linkage are phosphorothioate internucleoside linkages.

In some embodiments, the contiguous nucleotide sequence comprises LNA nucleosides and DNA nucleosides and 2′-O-methyl RNA nucleosides, and the internucleoside linkages between each of the nucleosides of the contiguous nucleotide linkage are phosphorothioate internucleoside linkages.

In some embodiments, the contiguous nucleotide sequence comprises 2′-O-methoxyethyl (2′MOE) nucleosides.

In some embodiments, the contiguous nucleotide sequence comprises 2′-O-methoxyethyl (2′MOE) nucleosides and DNA nucleosides.

Advantageously, the 3′ most nucleoside of the antisense oligonucleotide, or contiguous nucleotide sequence thereof is a 2′ sugar modified nucleoside.

Advantageously, the antisense oligonucleotide comprises at least one modified internucleoside linkage, such as phosphorothioate or phosphorodithioate.

In some embodiments, at least one internucleoside linkage in the contiguous nucleotide sequence is a phosphorothioate internucleoside linkages.

In some embodiments, at least one internucleoside linkage in the contiguous nucleotide sequence is a phosphorodithioate internucleoside linkages.

In some embodiments, at least one internucleoside linkage in the contiguous nucleotide sequence is a phosphodiester internucleoside linkages.

In some embodiments, all the internucleoside linkages within the contiguous nucleotide sequence are phosphorothioate internucleoside linkages.

In some embodiments, at least 75% the internucleoside linkages within the antisense oligonucleotide, or contiguous nucleotide sequence thereof, are phosphorothioate internucleoside linkages.

In some embodiments, all the internucleoside linkages within the antisense oligonucleotide, or contiguous nucleotide sequence thereof, are phosphorothioate internucleoside linkages.

In an advantageous embodiment of the invention the antisense oligonucleotide of the invention is capable of recruiting RNase H, such as RNase H1. In some embodiments, the antisense oligonucleotide of the invention, or the contiguous nucleotide sequence thereof is a gapmer.

In some embodiments, the antisense oligonucleotide, or contiguous nucleotide sequence thereof, consists or comprises a gapmer of formula 5′-F-G-F′-3′.

In some embodiments, region G consists of 6-16 DNA nucleosides.

In some embodiments, region F and F′ each comprise at least one LNA nucleoside.

### Pharmaceutically Acceptable Salts

In a further aspect, the invention provides a pharmaceutically acceptable salt of the antisense oligonucleotide or a conjugate thereof, such as a pharmaceutically acceptable sodium salt, ammonium salt or potassium salt.

### Method of Manufacture

In a further aspect, the invention provides methods for manufacturing the antisense oligonucleotides of the invention comprising reacting nucleotide units and thereby forming covalently linked contiguous nucleotide units comprised in the antisense oligonucleotide. Preferably, the method uses phophoramidite chemistry (see for example Caruthers et al, 1987, Methods in Enzymology vol. 154, pages 287-313). In a further embodiment, the method further comprises reacting the contiguous nucleotide sequence with a conjugating moiety (ligand) to covalently attach the conjugate moiety to the antisense oligonucleotide. In a further aspect, a method is provided for manufacturing the composition of the invention, comprising mixing the antisense oligonucleotide or conjugated antisense oligonucleotide of the invention with a pharmaceutically acceptable diluent, solvent, carrier, salt and/or adjuvant.

### Pharmaceutical Composition

In a further aspect, the invention provides pharmaceutical compositions comprising any of the aforementioned antisense oligonucleotides and/or antisense oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments, the pharmaceutically acceptable diluent is sterile phosphate buffered saline or sterile sodium carbonate buffer.

In some embodiments, the antisense oligonucleotide of the invention is in the form of a solution in the pharmaceutically acceptable diluent, for example dissolved in PBS or sodium carbonate buffer. In some embodiments, the antisense oligonucleotide of the invention, or pharmaceutically acceptable salt thereof is in a solid form, such as a powder, such as a lyophilized powder. In some embodiments, the antisense oligonucleotide may be pre-formulated in the solution or in some embodiments, may be in the form of a dry powder (e.g. a lyophilized powder) which may be dissolved in the pharmaceutically acceptable diluent prior to administration.

Suitably, for example the antisense oligonucleotide may be dissolved in a concentration of 0.1-100 mg/ml, such as 1-10 mg/the pharmaceutically acceptable diluent.

In some embodiments, the oligonucleotide of the invention is formulated in a unit dose of between 0.5-100 mg, such as 1 mg-50 mg, or 2-25 mg.

In some embodiments, the antisense oligonucleotide is used in the pharmaceutically acceptable diluent at a concentration of 50-300 μM.

Antisense oligonucleotides or antisense oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Pharmaceutical compositions, such as solutions, may be sterilized by conventional sterilization techniques, or may be sterile filtered. The resulting solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the antisense oligonucleotide or antisense oligonucleotide conjugate of the invention is a prodrug. In particular with respect to antisense oligonucleotide conjugates the conjugate moiety is cleaved off the antisense oligonucleotide once the prodrug is delivered to the site of action, e.g. the target cell.

### Applications

The antisense oligonucleotides of the invention may be utilized as research reagents for, for example, diagnostics, therapeutics and prophylaxis.

In research, such antisense oligonucleotides may be used to specifically modulate the synthesis of protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein.

If employing the antisense oligonucleotide of the invention in research or diagnostics the target nucleic acid may be a cDNA or a synthetic nucleic acid derived from DNA or RNA.

The present invention provides an in vivo or in vitro method for modulating gene expression in a target cell which is expressing a target protein, said method comprising administering an antisense oligonucleotide of the invention in an effective amount to said cell.

In some embodiments, the target cell, is a mammalian cell in particular a human cell. The target cell may be an in vitro cell culture or an in vivo cell forming part of a tissue in a mammal. In preferred embodiments, the target cell is present in the central nervous system.

### Therapeutic Applications

The antisense oligonucleotides of the invention, or the antisense oligonucleotide conjugates, salts or pharmaceutical compositions of the invention, may be administered to an animal or a human for the prevention or treatment of neurological disorders.

The neurological disorder which may be treated with the antisense oligonucleotides of the invention, or the antisense oligonucleotide conjugates, salts or pharmaceutical compositions of the invention may be Amyotrophic lateral sclerosis (ALS), Alzheimer's disease, Aneurysm, Back pain, Bell's palsy, Birth defects of the brain and spinal cord, Brain injury, Brain tumor, Cerebral palsy, Chronic fatigue syndrome, Concussion, Dementia, Disk disease of neck and lower back, Dizziness, Epilepsy, Guillain-Barré syndrome, Headaches and migraines, Multiple sclerosis, Muscular dystrophy, Neuralgia Neuropathy, Neuromuscular and related diseases, Parkinson's disease, Psychiatric conditions (severe depression, obsessive-compulsive disorder), Scoliosis, Seizures, Spinal cord injury, Spinal deformity and disorders, Spine tumor, Stroke and Vertigo which may be prevented, treated or ameliorated using the antisense oligonucleotide, antisense oligonucleotide conjugate, pharmaceutical composition or salt of the invention.

The invention provides for the antisense oligonucleotide, antisense oligonucleotide conjugate, composition or salt of the invention for the use for the prevention or for the treatment of the above recited neurological disorders.

The invention further relates to use of an antisense oligonucleotides, antisense oligonucleotide conjugate or a pharmaceutical composition of the invention for the manufacture of a medicament for the treatment or prevention of the above-recited neurological disorders.

The invention provides for the antisense oligonucleotide, antisense oligonucleotide conjugate, pharmaceutical composition or salt of the invention for the use as a medicament.

The invention provides for the use of the antisense oligonucleotide, antisense oligonucleotide conjugate, pharmaceutical composition or salt of the invention for manufacture of a medicament.

The invention provides for the antisense oligonucleotide, antisense oligonucleotide conjugate, pharmaceutical composition or salt of the invention for the use for the prevention or for the treatment of the above-recited neurological disorders.

The invention further relates to use of an antisense oligonucleotide, antisense oligonucleotide conjugate or a pharmaceutical composition of the invention for the manufacture of a medicament for the treatment or prevention of the above-recited neurological disorders.

### Method of Treatments

The invention provides methods for treating or preventing neurological disorder in a subject, such as a human, who is suffering from or is likely to suffer the above recited neurological disorders, comprising administering a therapeutically or prophylactically effective amount of an antisense oligonucleotide, an antisense oligonucleotide conjugate or a pharmaceutical composition of the invention to a subject who is suffering from or is susceptible to suffering from the above-recited neurological disorders.

By way of example, the method of treatment may be in subjects whose are suffering from an indication selected from the group consisting of the above-recited neurological disorders.

The method of the invention may be for treating the above-recited neurological disorders.

The methods of the invention are preferably employed for treatment or prophylaxis against the above recited neurological disorders.

### Administration

The antisense oligonucleotide, antisense oligonucleotide conjugate or pharmaceutical composition of the present invention may be administered via parenteral administration.

In some embodiments, the administration route is subcutaneous or intravenous.

In some embodiments, the administration route is selected from the group consisting of intravenous, subcutaneous, intra-muscular, intracerebral, epidural, intracerebroventricular intraocular, intrathecal administration, and transforaminal administration.

In some embodiments, the antisense oligonucleotide, antisense oligonucleotide conjugate or pharmaceutical composition of the present invention targets the brain, i.e. is delivered to the brain.

In some advantageous embodiments, the administration is via intrathecal administration, or epidural administration or transforaminal administration.

Advantageously, the antisense oligonucleotide, antisense oligonucleotide conjugate or pharmaceutical compositions of the present invention are administered intrathecally.

The invention also provides for the use of the antisense oligonucleotide of the invention, or antisense oligonucleotide conjugate thereof, such as pharmaceutical salts or compositions of the invention, for the manufacture of a medicament for the prevention or treatment of neurological disorder wherein the medicament is in a dosage form for intrathecal administration.

The invention also provides for the use of the antisense oligonucleotide or antisense oligonucleotide conjugate of the invention as described for the manufacture of a medicament for the manufacture of a medicament for the prevention or treatment of neurological disorder wherein the medicament is in a dosage form for intrathecal administration.

The invention also provides for the antisense oligonucleotide of the invention, or antisense oligonucleotide conjugate thereof, such as pharmaceutical salts or compositions of the invention, for use as a medicament for the prevention or treatment of neurological disorder wherein the medicament is in a dosage form for intrathecal administration.

The invention also provides for the antisense oligonucleotide or antisense oligonucleotide conjugate of the invention, for use as a medicament for the prevention or treatment of neurological disorder wherein the medicament is in a dosage form for intrathecal administration.

### Combination Therapies

In some embodiments, the antisense oligonucleotide, antisense oligonucleotide conjugate or pharmaceutical composition of the invention is for use in a combination treatment with another therapeutic agent. The therapeutic agent can for example be the standard of care for the diseases or disorders described above. In some embodiments, the compound of the invention is used in combination with small molecule analgesics which may be administered concurrently or independently of the administration of the compound or compositions of the invention. An advantage of a combination therapy of the compounds of the invention with small molecule analgesics is that small molecule analgesics have a rapid onset of neurological disorder relieving activity, typically with a short duration of action (hours-days), whereas the compounds of the invention has a delayed onset of activity (typically a few days or even a week+), but with a long duration of action (weeks-months, e.g. 2+, 3+ or 4 months+).

### EXAMPLES

### Example A: Synthesis of Oligomeric Compounds

Single-stranded oligonucleotides were synthesized using standard phosphoramidite chemistry. Non-modified DNA phosphoramidites and all standard reagents were purchased from Merck KGaA (Darmstadt, Germany). LNA phosphoramidites were produced in house (LNA phosphoramidites are also commercially available, e.g. Merck KGaA). 8-amino-dG, 8-oxo-dG, 7-Deaza-8-aza-dG (PPG) and 2′-deoxyinosine phosphoramidites were purchased from Glen Research (Sterling, Va.).

Oligonucleotides were synthesized on NittoPhase HL UnyLinker 350 support (Kinovate, Oceanside, Calif.) on an MerMade 12 (LGC BioAutomation, Irving, Tex.) at 20 μmol scale. After synthesis, the oligonucleotides were cleaved from the support using aqueous ammonia at 65° C. overnight. The oligonucleotides were purified by ion exchange on SuperQ-5PW gel (Tosoh Bioscience, Griesheim, Germany) using 10 mM NaOH buffers and a gradient of 2M NaCl, and desalted using a Millipore membrane. After lyophilization, the compounds were finally characterized by liquid chromatography-mass spectrometry (reverse phase and electrospray ionization-mass spectrometry).

### Example 1: In Vivo Study of Oligonucleotides Comprising Guanosine Analogues in Mice

In this example gapmer antisense oligonucleotides comprising natural guanosine or guanosine analogues were studied for their neurotoxicity in the following acute neurotoxicity assay:

Nine groups of six C57BL/6 black mice per group were injected via intracerebroventricular injection with a single dose of 100 μg of antisense oligonucleotides.

Mice of group 1 were injected with saline. Mice of group 2 where injected with SEQ ID No. 1 (control). Mice of groups 3 to 9 were respectively injected with SEQ ID No. 2 to 8. The behavior of the mice after injection was observed, reported and at 30 min, 1 h, 24 h and 14 days according to the following behavioral scoring categories (0-4):

A. “Hyperactivity, stereotypies and home cage behavior”

B. “Decreased vigilance, exploration and responsiveness”

C. “Motor coordination and strength”

D. “Posture, appearance and breathing”

E. “Tremor, hyperactivity, convulsion”

Typically, high neurotoxicity is seen as ataxia, convulsion and seizures within 30 min after injection.

TABLE 1
g^{PPG}: 7-Deaza-8-aza-deoxyguanosine

| | | | | | | | | | | Median | |
|---|---|---|---|---|---|---|---|---|---|---|---|
| SEQ | | | | | | | | | | time to | Euthanized |
| No. | | 30 min | | 1 hour | | 24 hours | | 14 days | | return to | on ethical |
| ID | | score | N.a. | score | N.a. | score | N.a. | score | N.a. | baseline | reasons |
| | Saline | 0 | 6 | 0 | 6 | 0 | 6 | 0 | 6 | 0.5 | 0 |
| 1 | CcAAAtcttataataACtA | 0 | 6 | 0 | 6 | 0 | 6 | 0 | 6 | 0.5 | 0 |
| | C (non toxic control) | | | | | | | | | | |
| 2 | CTCAtacttgctttaAT | 3.7 | 2 | 3.8 | 3 | 0 | 4 | 0 | 4 | 0.8 | 2 |
| 3 | CTCAtacttIctttaAT | 5.8 | 0 | 4.7 | 1 | 0 | 5 | 0 | 5 | 4 | 1 |
| 4 | CTCAacttg^{oxo}ctttaAT | 0.3 | 4 | 0 | 6 | 0 | 6 | 0 | 6 | 0.5 | 0 |
| 5 | CTCAtacttg^{N}ctttaAT | 3.8 | 1 | 3.3 | 1 | 0 | 5 | 0 | 5 | 3 | 1 |
| 6 | CTCAtacttg^{PPG}ctttaAT | 1.3 | 3 | 0.2 | 5 | 0 | 6 | 2.7 | 2 | 0.8 | 0 |
| 7 | TTTTaaccagagtggCAT | 8.2 | 0 | 0 | 0 | 0 | 0 | 0 | 0 | — | 6 |
| 8 | TTTTaaccagagtggcaTC | 8.7 | 0 | 0 | 0 | 0 | 0 | 0 | 0 | — | 6 |

Capital letters in these sequences indicate nucleoside that have an LNA modified ribose.
All LNA C are 5-methyl cytosine
Small letters in these sequences indicate DNA.
N.a.: nothing abnormal
I: inosine nucleotide 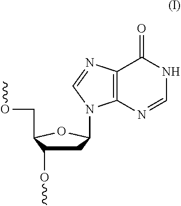
g^{N}: 8-amino-dG: 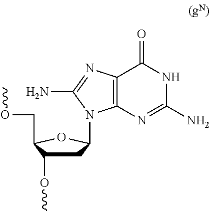
g^{OXO}: 8-Oxo-deoxyguanosine

The following modified guanosine nucleosides were used in these sequences:

This study shows:
Sequence (SEQ ID No 1., control) without g show no neurotoxicity
Sequences with two or three g (SEQ ID No. 7 and 8) show a relative higher neurotoxicity compared to sequences with one g (SEQ ID No. 2)
Sequence with g (SEQ ID No. 2) shows neurotoxicity
Sequence with I (SEQ ID No. 3) shows neurotoxicity

### Conclusion:

many g in the sequence: relative high neurotoxicity
modified g in the sequence show relative lower neurotoxicity compared to sequences with non modified g.
g^{oxo} and g^{PPG} in the sequence show the least relative lower neurotoxicity

Acute neurotox is groups 7 and 8 lead to euthanasia

Suggest that g are toxic, many g more toxic, modified g less toxic.

### Example 2

Following the procedure of example 1, the gapmer compounds of table 2 were tested on 8 groups of 6 C57BL/6 mice. Results are shown in table 2.

TABLE 2

| | | | | | | | | | | Median | |
|---|---|---|---|---|---|---|---|---|---|---|---|
| | | | | | | | | | | time to | |
| SEQ | | | | | | | | | | return | Euthanized |
| ID | | 30 min | | 1 hour | | 24 hours | | 14 days | | to | on ethical |
| No. | | score | N.a. | score | N.a. | score | N.a. | score | N.a. | baseline | reasons |
| — | Saline | 0 | 6 | 0 | 6 | 0 | 6 | 0 | 6 | 0.5 | 0 |
| 1 | CcAAAtcttataataACtAC (non toxic | 0 | 6 | 0 | 6 | 0 | 6 | 0 | 6 | 0.5 | 0 |
| | control) | | | | | | | | | | |
| 9 | CTAcatctcatactTgC | 4.7 | 0 | 4.5 | 3 | 0 | 4 | 0 | 4 | 1 | 2 |
| 10 | CTAcatctcatactTg^{PPG}C | 0.2 | 5 | 0 | 6 | 0 | 6 | 0 | 6 | 0.5 | 0 |
| 11 | CTAcatctcatactTg^{oxo}C | 0 | 6 | 0 | 6 | 0 | 6 | 0 | 6 | 0.5 | 0 |
| 12 | CTAcatctcatactTIC | 3.3 | 2 | 3.5 | 3 | 0 | 5 | 0 | 5 | 1 | 1 |
| 13 | CTAcatctcatactTg^{N}C | 8.3 | 0 | 9.7 | 0 | 0 | 0 | 0 | 0 | — | 6 |

Capital letters in these sequences indicate nucleoside that have an LNA modified ribose. All LNA C are 5-methyl cytosine
Small letters in these sequences indicate DNA.
N.a.: nothing abnormal
I: inosine nucleoside
The following modified guanosine nucleosides were used in these sequences:
g^{PPG}: 7-Deaza-8-aza-deoxyguanosine
g^{N}: 8-amino-dG
g^{oxo}: 8-Oxo-deoxyguanosine

This study shows that a sequence without guanosine shows no neurotoxicity, see sequence (SEQ ID No 1, control). The sequence with g^{PPG} (SEQ ID No. 10) shows much less neurotoxicity compared to the same sequences with a natural guanosine (SEQ ID No. 9). The sequence with g^{oxo} (SEQ ID No. 11) shows no neurotoxicity. It can be compared to the same sequences with a natural guanosine (SEQ ID No. 9) and a g^{PPG} (SEQ ID No. 10). The sequence with I (SEQ ID No. 12) shows relative higher neurotoxicity than the same sequences with guanosine. The sequence with g^{N} (SEQ ID No. 13) shows relative higher neurotoxicity than the same sequences with guanosine.

Conclusion: the inventors were surprised to find that the proportion of natural, i.e. unmodified guanosine nucleobases within a polynucleotide sequence was directly correlated to the likelihood that a polynucleotide, such as an antisense oligonucleotide, is neurotoxic. Such neurotoxicity shows to be acute and fatal. Replacement of natural guanosine by guanosine analogues g^{PPG} and g^{oxo} provided for a reduce neurotoxicity.

### Example 3

Following the procedure of example 1, the gapmer compounds of table 2 were tested on 9 groups of 6 C57BL/6 mice. Results are shown in table 3.

TABLE 3

| | | | | | | | | | | | Median | |
|---|---|---|---|---|---|---|---|---|---|---|---|---|
| SEQ | | | | | | | | | | | time to | Euthanized |
| ID | | | 30 min | | 1 hour | | 24 hours | | 14 days | | return to | on ethical |
| No. | | Dose | score | N.a. | score | N.a. | score | N.a. | score | N.a. | baseline | reasons |
| | Saline | | 0 | 6 | 0 | 6 | 0 | 6 | 0 | 6 | 0.5 | 0 |
| 1 | CcAAAtcttataataACtAC | 50 μg | 0 | 6 | 0 | 6 | 0 | 6 | 0 | 6 | 0.5 | 0 |
| | (non toxic control) | | | | | | | | | | | |
| 1 | CcAAAtcttataataACtAC | 100 μg | 1.2 | 0 | 0 | 6 | 0 | 6 | 0 | 6 | 1.0 | 0 |
| | (non toxic control) | | | | | | | | | | | |
| 14 | ACAggattagttCTA | 50 μg | 1.5 | 0 | 0.7 | 2 | 0 | 6 | 0 | 6 | 2.0 | 0 |
| 14 | ACAggattagttCTA | 100 μg | 5.3 | 0 | 6.0 | 0 | 0 | 6 | 0 | 6 | 4.0 | 0 |
| 15 | ACAg^{oxo}g^{oxo}attag^{oxo}ttCT | 50 μg | 1.0 | 1 | 0 | 6 | 0 | 6 | 0 | 6 | 1.0 | 0 |
| | A | | | | | | | | | | | |
| 15 | ACAg^{oxo}g^{oxo}attag^{oxo}ttCT | 100 μg | 0.7 | 2 | 0 | 6 | 0 | 6 | 0 | 6 | 1.0 | 0 |
| | A | | | | | | | | | | | |
| 16 | ACAg^{PPG}g^{PPG}attag^{PPG}ttC | 50 μg | 0.2 | 5 | 0 | 6 | 0 | 6 | 0 | 6 | 0.5 | 0 |
| | TA | | | | | | | | | | | |
| 16 | ACAg^{PPG}g^{PPG}attag^{PPG}ttC | 100 μg | 0.2 | 5 | 0 | 6 | 0 | 6 | 0 | 6 | 0.5 | 0 |
| | TA | | | | | | | | | | | |

Capital letters in these sequences indicate nucleoside that have an LNA modified ribose. All LNA C are 5-methy cytosine
Small letters in these sequences indicate DNA.
N.a.: nothing abnormal
I: inosine nucleoside
The following modified guanosine nucleosides were used in these sequences:
g^{PPG}: 7-Deaza-8-aza-deoxyguanosine
g^{N}: 8-amino-dG
g^{oxo}: 8-Oxo-deoxyguanosine

This study reveals that the same nucleotide sequence with three natural guanosine in the gap (SEQ ID No. 14), which was tested at 50 μg and 100 μg show a dose dependent increase in neurotoxicity. The same nucleotide sequence (SEQ ID. No. 15) as gapmer of SEQ ID No. 14, but with three guanosine analogues (g^{oxo}) were tested at 50 μg and 100 μg and show a relative much lower neurotoxicity compared to the gapmer of SEQ ID No. 14. Interestingly there is no increase in neurotoxicity when increasing the dose from 50 μg to 100 μg.

The same nucleotide sequence (SEQ ID. No. 16) as gapmer of SEQ ID No. 14, but with three guanosine analogues (g^{PPG}) were tested at 50 μg and 100 μg and show a relative much lower neurotoxicity compared to the gapmer of SEQ ID No. 14. Interestingly there is no increase in neurotoxicity when increasing the dose from 50 μg to 100 μg.

1. A polynucleotide that comprises:
at least one phosphorothioate internucleoside linkage and
at least a guanosine analogue comprising a guanine analogue selected from the group consisting of:

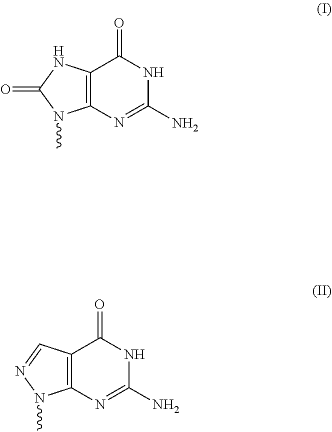

2. The polynucleotide of claim 1 wherein it is single stranded.

3. The polynucleotide of claim 2 wherein it is an antisense oligonucleotide.

4. The polynucleotide of claim 1 wherein it is double stranded.

5. The polynucleotide of claim 4 wherein it is an siRNA or shRNA.

6. The polynucleotide of claim 1, further comprising one or more 2′ sugar modified nucleosides.

7. The polynucleotide of claim 2, wherein the 2′ sugar modified nucleoside(s) are independently selected from the group consisting of locked nucleic acids and 2′ sugar substituted nucleosides.

8. The polynucleotide of claim 6, wherein one or more of the 2′ sugar modified nucleosides are locked nucleic acid selected from the group consisting of: 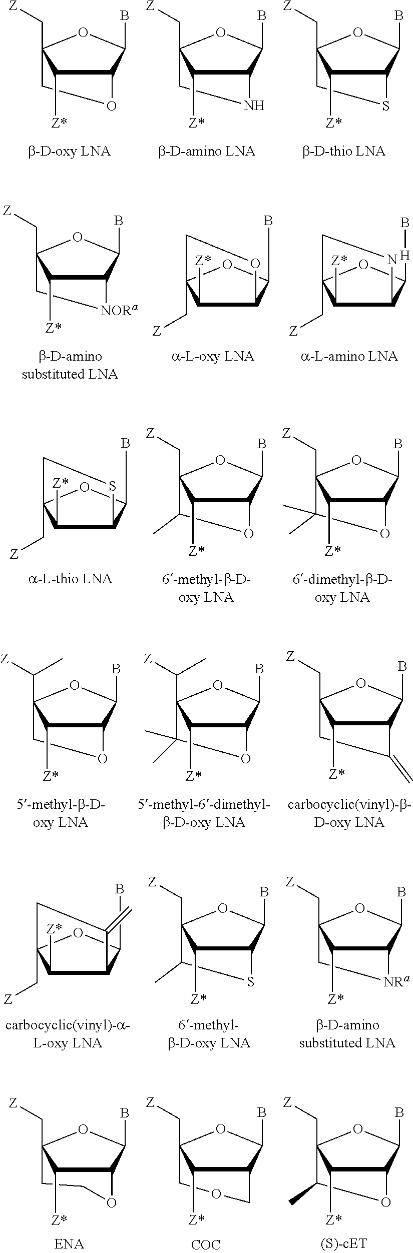
wherein B is a natural or modified nucleobase and Z is an internucleoside linkage to an adjacent nucleoside or a 5′-terminal group and Z* is an internucleoside linkage to an adjacent nucleoside or a 3′-terminal group.

9. The polynucleotide of claim 6, wherein one or more 2′ sugar modified nucleosides are selected from the group consisting of:

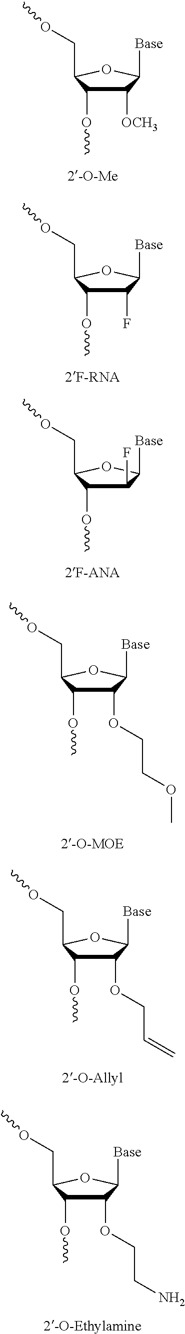

10. The polynucleotide of claim 1, wherein the guanosine analogue is selected from the group consisting of: 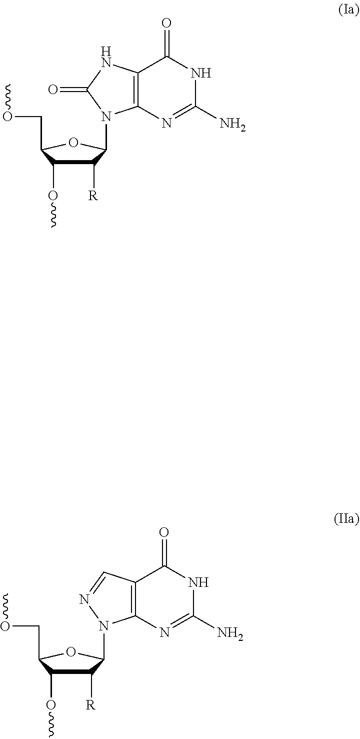
wherein R is H or OH

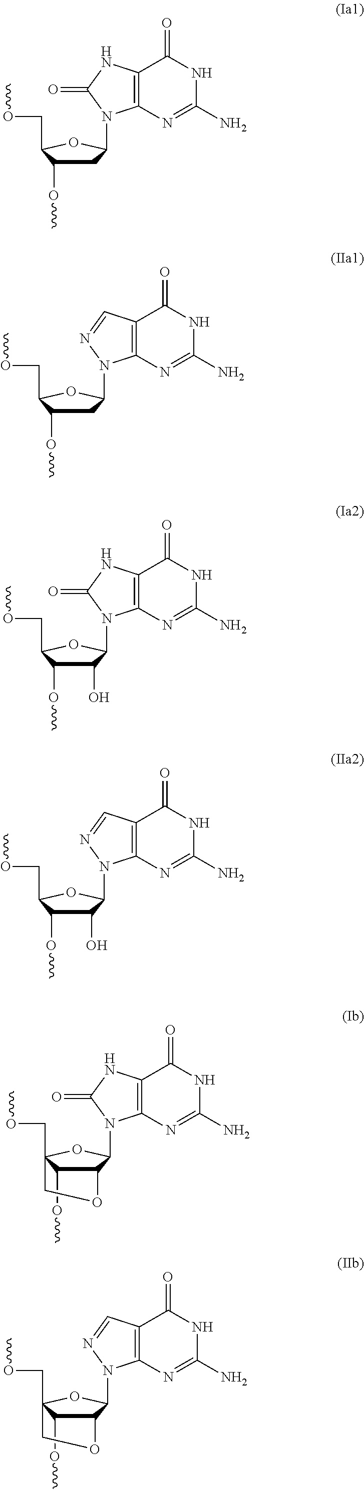

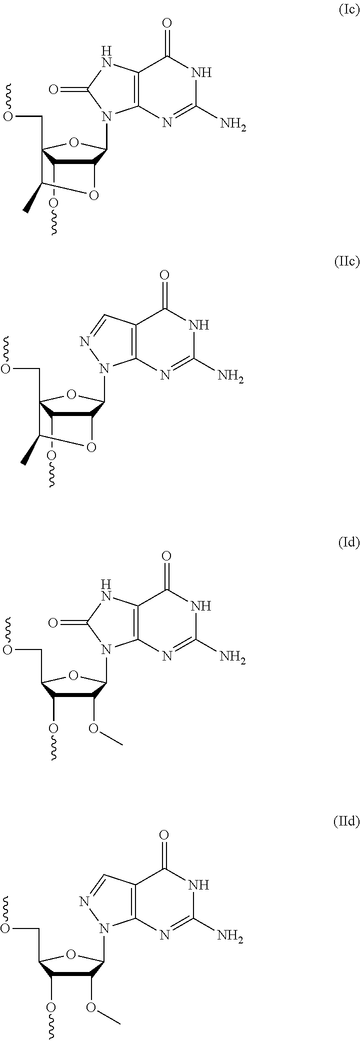

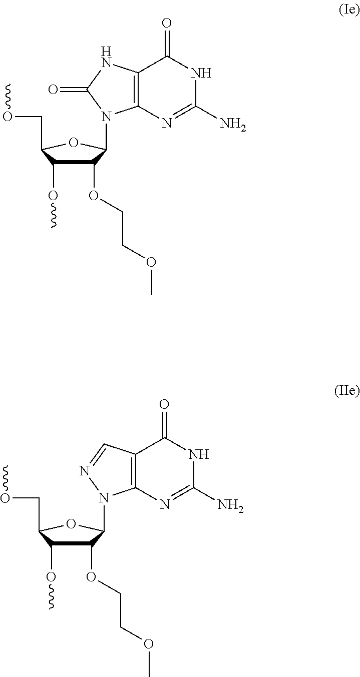

11. The polynucleotide of claim 10 wherein the guanosine analogue is (Ia).

12. The polynucleotide of claim 10 wherein the guanosine analogue is (Ia1).

13. The polynucleotide of claim 10 wherein the guanosine analogue is (Ia2).

14. The polynucleotide of claim 10 wherein the guanosine analogue is (Ib).

15. The polynucleotide of claim 10 wherein the guanosine analogue is (IIa).

16. The polynucleotide of claim 10 wherein the guanosine analogue is (IIa1).

17. The polynucleotide of claim 10 wherein the guanosine analogue is (IIa2).

18. The polynucleotide of claim 10 wherein the guanosine analogue is (IIb).

19. The polynucleotide of claim 10 wherein the guanosine analogue is (Ic).

20. The polynucleotide of claim 10 wherein the guanosine analogue is (IIc).

21. The polynucleotide of claim 10 wherein the guanosine analogue is (Id).

22. The polynucleotide of claim 10 wherein the guanosine analogue is (IId).

23. The polynucleotide of claim 10 wherein the guanosine analogue is (Ie).

24. The polynucleotide of claim 10 wherein the guanosine analogue is (He).

25. The polynucleotide of claim 2, wherein the antisense oligonucleotide is a gapmer.

26. The polynucleotide of claim 2, wherein it comprises at least one additional nucleoside that has a modified ribose and wherein the wherein the ribose modification is selected from the group consisting of locked nucleic acid or 2′ modification.

27. The polynucleotide of claim 25, wherein the guanosine analogue is in the gap region of the gapmer and is of formula Ia or IIb and wherein R is H.

28. The polynucleotide of claim 25, wherein the guanosine analogue is not in the flank of the gapmer.

29. The polynucleotide of claim 1, wherein it comprises one guanosine analogue.

30. The polynucleotide of claim 1, wherein it comprises two guanosine analogues.

31. The polynucleotide of claim 1, wherein it comprises three guanosine analogues.

32. The polynucleotide of claim 1, wherein it comprises no natural guanosine.

33. The polynucleotide of claim 1, wherein it is selected from the group consisting of:
(SEQ ID No. 4)
CTCAacttg^{OXO}ctttaAT;
(SEQ ID No. 5)
CTCAtacttg^{N}ctttaAT;
(SEQ ID No. 6)
CTCAtacttg^{PPG}ctttaAT;
(SEQ ID No. 9)
CTAcatctcatactTgC;
(SEQ ID No. 10)
CTAcatctcatactTg^{PPG}C;
(SEQ ID No. 11)
CTAcatctcatactTg^{OXO}C;
(SEQ ID No. 13)
CTAcatctcatactTg^{N}c;
(SEQ ID No. 15)
ACAg^{OXO}g^{OXO}attag^{OXO}ttCTA;
and
(SEQ ID No. 16)
ACAg^{PPG}g^{PPG}attag^{PPG}ttCTA;
wherein capital letters in these sequences indicate nucleoside that have an LNA modified ribose, all LNA C are 5-methyl cytosine and small letters in these sequences indicate DNA,
g^{PPG} is 7-Deaza-8-aza-deoxyguanosine,
g^{N} is 8-amino-dG, and
g^{oxo} is 8-Oxo-deoxyguanosine.

34. A polynucleotide of claim 1 for use as a medicament.

35. The polynucleotide according to claim 34 wherein is for administration to the central nervous system, or for treatment of a neurological disorder, such as a CNS disorder selected from the group consisting of amyotrophic lateral sclerosis (ALS), Angelman's, Alzheimer's disease, Aneurysm, Back pain, Bell's palsy, Birth defects of the brain and spinal cord, Brain injury, Brain tumor, Cerebral palsy, Chronic fatigue syndrome, Concussion, Dementia, Disk disease of neck and lower back, Dizziness, Epilepsy, Guillain-Barré syndrome, Headaches and migraines, Multiple sclerosis, Muscular dystrophy, Neuralgia, Neuropathy, Neuromuscular and related diseases, Parkinson's disease, Psychiatric conditions (severe depression, obsessive-compulsive disorder), Scoliosis, Seizures, Spinal cord injury, Spinal deformity and disorders, Spine tumor, Stroke and Vertigo.

36. The polynucleotide according to claim 35, wherein it is for administration via intrathecal injection.

37. The polynucleotide of claim 1 for use as a medicament to treat a medical condition wherein modulation of Ube3A is beneficial, such as for the treatment of Angelman's.

38. The polynucleotide of claim 1 for use as a medicament to treat a medical condition wherein modulation of ATXN2 is beneficial.

39. The polynucleotide of claim 1 for use as a medicament to treat a medical condition wherein modulation of ATXN3 is beneficial.

40. The polynucleotide of claim 1, wherein it is used as a medicament wherein reduced neurotoxicity is needed.

41. A method for synthesizing a polynucleotide with a reduced toxicity of claim 1, said method comprising coupling a nucleotide monomer, such as a phosphoramidite to a further nucleotide, or an oligonucleotide, wherein the nucleotide monomer comprises a guanosine analogue.

42. A method for selecting a polynucleotide with a reduced toxicity over a reference polynucleotide, wherein the polynucleotide of claim 1 and wherein the reference polynucleotide and the less neurotoxic antisense oligonucleotide have the same nucleotide sequence and comprise at least one guanosine with the difference that the less neurotoxic polynucleotide comprises at least a guanosine analogue compared to the reference polynucleotide.

43. Use of a compound containing a guanosine analogue selected from the group consisting of (Ia), (Ib), (Ic), (Id), (Ie), (IIa), (IIb), (IIc), (IId) and (IIe) in the manufacture of a polynucleotide.

44. A method for up-regulating Ube3a expression in a target cell which is expressing Ube3a-ATS, said method comprising administering the polynucleotide of claim 1 which targets Ube3a-ATS, in an effective amount to said cell.

45. The method according to claim 38, wherein said method is an in vivo method or an in vitro method.

46. A method for treating or preventing neurological disorder in a subject, a human, who is suffering from or is likely to suffer neurological disorders, comprising administering a therapeutically or prophylactically effective amount of the polynucleotide of claim 1, to prevent or alleviate the neurological disorder.


SEQUENCE LISTING (16 sequences)

SEQ ID NO: 1 - DNA, 20 bp, Artificial Sequence
  Antisense oligonucleotide
ccaaatcttataataactac

SEQ ID NO: 2 - DNA, 17 bp, Artificial Sequence
  antisense oligonucleotide
ctcatacttgctttaat

SEQ ID NO: 3 - DNA, 17 bp, Artificial Sequence
  antisense oligonucleotide
  misc_feature (10)..(10): n is Inosine
ctcatacttnctttaat

SEQ ID NO: 4 - DNA, 16 bp, Artificial Sequence
  antisense oligonucleotide
ctcaacttgctttaat

SEQ ID NO: 5 - DNA, 17 bp, Artificial Sequence
  antisense oligonucleotide
ctcatacttgctttaat

SEQ ID NO: 6 - DNA, 17 bp, Artificial Sequence
  antisense oligonucleotide
ctcatacttgctttaat

SEQ ID NO: 7 - DNA, 18 bp, Artificial Sequence
  antisense oligonucleotide
ttttaaccagagtggcat

SEQ ID NO: 8 - DNA, 19 bp, Artificial Sequence
  antisense oligonucleotide
ttttaaccagagtggcatc

SEQ ID NO: 9 - DNA, 17 bp, Artificial Sequence
  antisense oligonucleotide
ctacatctcatacttgc

SEQ ID NO: 10 - DNA, 17 bp, Artificial Sequence
  antisense oligonucleotide
ctacatctcatacttgc

SEQ ID NO: 11 - DNA, 17 bp, Artificial Sequence
  antisense oligonucleotide
ctacatctcatacttgc

SEQ ID NO: 12 - DNA, 17 bp, Artificial Sequence
  antisense oligonucleotide
  misc_feature (16)..(16): n is Inosine
ctacatctcatacttnc

SEQ ID NO: 13 - DNA, 17 bp, Artificial Sequence
  antisense oligonucleotide
ctacatctcatacttgc

SEQ ID NO: 14 - DNA, 15 bp, Artificial Sequence
  antisense oligonucleotide
acaggattagttcta

SEQ ID NO: 15 - DNA, 15 bp, Artificial Sequence
  antisense oligonucleotide
acaggattagttcta

SEQ ID NO: 16 - DNA, 15 bp, Artificial Sequence
  antisense oligonucleotide
acaggattagttcta